SEQUENCE LISTING (15 sequences)

SEQ ID NO: 1 - protein, 4 aa, Artificial Sequence
  cell adhesion molecule
RGDY

SEQ ID NO: 2 - protein, 7 aa, Artificial Sequence
  cell adhesion molecule
KQAGDVY

SEQ ID NO: 3 - protein, 5 aa, Artificial Sequence
  cell adhesion molecule
YIGSR

SEQ ID NO: 4 - protein, 5 aa, Artificial Sequence
  cell adhesion molecule
REDVY

SEQ ID NO: 5 - protein, 6 aa, Artificial Sequence
  cell adhesion molecule
IKVANY

SEQ ID NO: 6 - protein, 11 aa, Artificial Sequence
  cell adhesion molecule
RNIAEIIKDIY

SEQ ID NO: 7 - protein, 11 aa, Artificial Sequence
  cell adhesion molecule
KHIFSDDSSEY

SEQ ID NO: 8 - protein, 6 aa, Artificial Sequence
  cell adhesion molecule
VPGIGY

SEQ ID NO: 9 - protein, 8 aa, Artificial Sequence
  cell adhesion molecule
FHRRIKAY

SEQ ID NO: 10 - protein, 5 aa, Artificial Sequence
  cell adhesion molecule
KRSRY

SEQ ID NO: 11 - protein, 22 aa, Artificial Sequence
  cell adhesion molecule
NSPVNSKIPKACCVPTELSAIY

SEQ ID NO: 12 - protein, 5 aa, Artificial Sequence
  cell adhesion molecule
APGLY

SEQ ID NO: 13 - protein, 4 aa, Artificial Sequence
  cell adhesion molecule
VRNY

SEQ ID NO: 14 - protein, 10 aa, Artificial Sequence
  cell adhesion molecule
AAAAAAAAAY

SEQ ID NO: 15 - protein, 10 aa, Artificial Sequence
  cell adhesion molecule
GRGDGGGGGY

(19) United States
(12) Patent Application Publication
(10) Pub. No.: US 2013/0224795 A1
(43) Pub. Date: Aug. 29, 2013

(54) IMMOBILIZATION METHOD OF BIOACTIVE MOLECULES USING POLYPHENOL OXIDASE

(71) Applicants: Ki-Dong Park, Seoul (KR); Kyung-Min Park, Anyang-si (KR); Yoon-Ki Joung, Suwon-si (KR); Yun-Ki Lee, Seongnam-si (KR); Ji-Hye Oh, Changwon-si (KR); Seung-Mi Hyun, Incheon (KR)

(72) Inventors: Ki-Dong Park, Seoul (KR); Kyung-Min Park, Anyang-si (KR); Yoon-Ki Joung, Suwon-si (KR); Yun-Ki Lee, Seongnam-si (KR); Ji-Hye Oh, Changwon-si (KR); Seung-Mi Hyun, Incheon (KR)

(73) Assignee: Ajou University Industry-Academic Cooperation Foundation, Suwon-si (KR)

(21) Appl. No.: 13/882,539

(22) PCT Filed: Aug. 19, 2011
(86) PCT No.: PCT/KR11/06148

§ 371 Date: Apr. 30, 2013

(30) Foreign Application Priority Data
Nov. 1, 2010 (KR) 10 2010 0107782

(51) Int. Cl. C12P 21/00; C12P 13/02

(52) U.S. Cl. CPC C12P 21/00; C12P 13/02
USPC 435/68.1; 435/129

(57) ABSTRACT

A method is provided for immobilizing a bioactive molecule onto a surface using polyphenol oxidase. In the presence of polyphenol oxidase, a bioactive molecule containing a phenol or catechol group can be simply in situ oxidized within a short time to dopa or dopaquinone which forms a coordinate bond with a metal or polymer substrate, thus immobilizing the bioactive molecule onto the surface with stability. Based on the surface immobilization of bioactive molecules using polyphenol oxidase, various bioactive molecules such as osteogenetic peptides and growth factors can be simply immobilized to medical metal or polymer substrate surfaces such as orthopedic or dental implants which can be then effectively used to induce rapid osteogenesis after being transplanted. Also, antithrombotic agents and/or entothelialization inducing agents may be immobilized to medical substrates for vascular systems, such as stents and artificial blood vessels, thus guaranteeing hemocompatibility to the medical substrates.

### TECHNICAL FIELD

The present invention relates to an immobilization method of bioactive molecules using a polyphenol oxidase by which the bioactive molecules can be simply immobilized onto surfaces of various biomaterials for biomedical applications.

### BACKGROUND ART

With the aim of performing, augmenting or replacing a natural function of tissues which is lost by an accident for a congenital reason, biomaterials, whether produced in nature or synthesized by using chemical materials, are adopted intermittently or continually to the body, thus being in constant contact with body tissues and being exposed to body fluids.

For implantation into the body, biomaterials are required to have tissue-compatibility and hemo-compatibility responsible for tissue regenerative capacity and biological functionality. Requirements of biomaterials for use in tissue regeneration include tissue adhesiveness and tissue induction while biocompatibility, such as hemocompatibility and repression of non-specific adsorption, is the virtue of the biomaterials suitable for use in circulation devices or biosensors.

There have been many studies that are directed toward the improvement of metallic, polymeric or ceramic biomaterials in biofunctionality. Polymeric biomaterials, although relatively low in mechanical strength compared to other biomaterials, have the advantage of being readily chemically modified through various functional groups. In spite of being polymeric biomaterials, non-active polymers such as Teflon, silicon, etc., are however difficult to chemically modify.

To overcome these limitations, many studies of surface modification of biomaterials have been conducted. In fact, some of them have succeeded in industrial applications. Among typical techniques of surface chemical modification are plasma treatment, surface coating with polymers, and surface treatment with acid/base.

For dental implants, various implant surface treatments including titanium plasma dispersion, hydroxyapatite coating, acid/base corrosion, and immobilization of, or coating with, bioactive molecules have been developed so as to enhance osteogenesis around implant sites.

These surface treatment methods enlarge the surface area of the implant to promote interaction at bone-implant interfaces or guarantee the activity of bioactive molecules to facilitate osteogenesis around implant sites. However, these methods have the drawbacks of cracking coating surfaces, releasing metal ions, and providing habitats for bacteria.

As for the immobilization and coating technique of bioactive molecules, its disadvantage is the technical complexity. In addition, not only is the bioactive molecule to be immobilized on the surface limited in quantity, but it also takes a long period of time to achieve the chemical reactions for the immobilization. For example, 1-ethyl-3-[3-dimethylaminopropyl]carbodiimide chloride (EDC)/N-hydroxysulfosuccinimide (NHS), typical reagents for conjugation, require 3 to 24 hrs on average for coupling therewith. Further, remaining EDC-derived urea derivatives, which cause cytotoxicity, are difficult to remove. Moreover, the introduction of functional groups, e.g., carboxyl or amino groups, onto polymer surfaces may need a treatment with external energy, such as plasma or ion beams. In this case, a change in the morphology of the surface can be incurred.

For use in circulatory systems, biomaterials, such as stents or artificial vessels, must have excellent hemocompatibility (antithrombogenicity) on their surfaces. Stents must be supplemented with the ability to inhibit the proliferation of smooth muscle cells in addition to anti-thromobogenicity. Artificial vessels are required to have the biological functionality of inducing and regenerating vascular endothelial cells as well as anti-thrombogenicity.

To improve their biocompatibility, extensive research has been focused on polymer/drug coating technology and the immobilization of bioactive molecules. As mentioned above, however, conventional surface immobilization or coating technologies of metallic or polymeric materials suffer from the following disadvantages: technical complexity, a limitation in the amount of bioactive molecules available for immobilization, difficulty in removal remaining byproducts, and long reaction time for chemical coupling and coating.

Recently, surface immobilization and coating techniques have paid attention to 3,4-dihydroxyphenyl-L-alanine (DOPA). DOPA, an amino acid abundantly found in mussel foot adhesive proteins, forms a very strong hydrogen bond with hydrophilic surfaces and a strong coordinate covalent bond with metals or semi-metals. Being oxidized to dopaquinone, dopa residues function to crosslink protein molecules.

Surface immobilization techniques using dopa derivatives have been studied by the lab of Professor Cha, Postech University. The research team of Professor Cha developed a method of extracting mussel foot proteins and conducted a study on the improvement of polymeric or metallic biomaterials in biocompatibility which was directed toward the immobilization of a dopa-based cell adhesive protein prepared using the method of Cha onto polymer or metal surfaces through coordinate bonds.

In collaboration with Prof. Phillip B. Messersmith, Northwestern University, U.S. A., Prof. Lee Haeshin, KAIST, Korea, developed a method of reducing protein adsorption by PEGylation of metal surfaces with dopa derivatives.

Utilizing coordinate bonds between dopa molecules and metal or polymer surfaces, the dopa-based surface immobilization technique has the advantage over the physical coating technique in that immobilization material can be stably introduced. However, the dopa-based surface immobilization technique using dopa molecules requires a long reaction time for immobilization. For example, it takes 12 hrs or longer to conduct a reaction necessary for the immobilization of dopa-conjugated polyethylene glycol (PEG) onto a metal or polymer surface. In addition, another problem with the technique of surface immobilization with dopa derivatives is the formation of polydopa due to the oxidation of dopa molecules during the synthesis and the storage of the product. To solve this problem, a process of preventing dopa oxidation by adjusting pH is additionally required. Furthermore, this technique is difficult to apply for the backbone of a molecule or polymer containing a neucleophile such as amine or thiol because dopaquinones which result from the oxidation of dopa in the coating process as stated above may undergo neucleophilic attack by the amine or thiol group. Hence, not only must dopa molecules be prevented from oxidation by pH adjustment during their synthesis, but also they need caution for the storage thereof lest they undergo oxidation even after the synthesis.

Therefore, there is a need for a simple technique by which various materials including bioactive molecules can be stably immobilized onto metallic or polymeric biomaterials within a short time period.

### DISCLOSURE OF INVENTION

### Technical Problem

Leading to the present invention, intensive and thorough research into the immobilization of bioactive molecules on solid phases, conducted by the present inventors, aiming to avoid the problems encountered in the prior art, resulted in the finding that phenol or catechol molecules can be anchored onto a substrate surface by the enzymatic action of polyphenol oxidase (PPO).

It is an object of the present invention to provide a method by which phenol or catechol-containing bioactive molecules, such as cell adhesion peptides, growth factors, growth hormones, proteins, antithrombotic agents and the like, can be immobilized stably on metallic or polymeric substrates by a simple process such as immersion or spraying in the presence of polyphenol oxidase (PPO).

### Solution to Problem

In accordance with an aspect thereof, the present invention provides a method for the immobilization of a bioactive molecule on a surface, comprising: preparing a phenol- or catechol-containing bioactive molecule (step 1); and treating a substrate surface with the bioactive molecule in the presence of polyphenoloxidase to immobilize the bioactive molecule onto the surface.

By the action of polyphenol oxidase (PPO), the phenol- or catechol-containing bioactive molecule is in situ oxidized into dopa or dopaquinone which shows a strong surface adhesiveness. Dopa and dopaquinone molecules can be stably anchored to a metal or polymer surface through a coordinate bond.

In greater detail, polymers or metals such as stainless steel and titanium can be used as substrates on which a bioactive molecule is immobilized using polyphenol oxidase. First, a substrate is immersed in phosphate buffer saline to which a bioactive molecule of interest is then added. Thereafter, polyphenol oxidase is introduced into the solution.

In this context, the concentration of the immobilized bioactive molecule can be adjusted with reaction time and the concentrations of the polyphenol oxidase and initial bioactive molecule. After completion of the immobilization, the substrate may be washed three to five times with distilled water and dried in a vacuum oven. Thus, the substrate is found to have the bioactive molecule immobilized thereonto.

The bioactive molecule may be a cell adhesion peptide containing a tyrosine residue. Preferable examples of the cell adhesion peptide include peptides of SEQ ID NO. 1 (RGD-Y), SEQ ID NO. 2 (KQAGDV-Y), SEQ ID NO. 3 (YIGSR), SEQ ID NO. 4 (REDV-Y), SEQ ID NO. 5 (IKVAN-Y), SEQ ID NO. 6 (RNIAEIIKDI-Y), SEQ ID NO. 7 (KHIFSDDSSE-Y), SEQ ID NO. 8 (VPGIG-Y), SEQ ID NO. 9 (FHRRIKA-Y), SEQ ID NO. 10 (KRSR-Y), SEQ ID NO. 11 (NSPVNSKIPKACCVPTELSAI-Y), SEQ ID NO. 12 (APGL-Y), SEQ ID NO. 13 (VRN-Y) and SEQ ID NO. 14 (AAAAAAAAA-Y), but are not limited thereto.

Also, the bioactive molecule may comprise one or more polymers, represented by the following Chemical Formula 1, in which a phenol or catechol derivative is grafted via a linker or directly onto a polymer backbone:

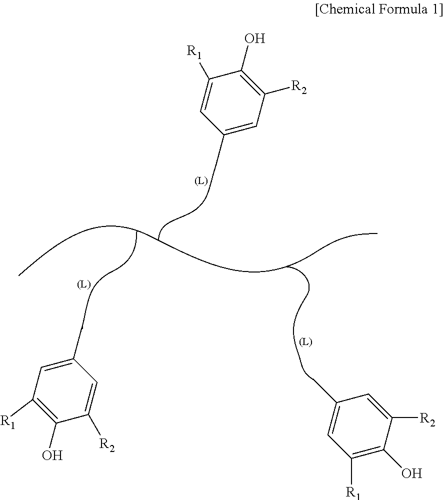

wherein, R₁ and R₂, which may be the same or different, are independently hydroxyl or hydrogen; and L is a polymeric linker.

For use in the present invention, a bioactive molecule is modified with a phenol or catechol group. In this regard, the polymer of Chemical Formula 1 may be prepared by grafting a phenol or catechol derivative represented by the following Chemical Formula 2 onto a polymer backbone having amino, hydroxyl or carboxyl groups through an amide, urethane, urea or ester bond, with a polymer serving as a linker:

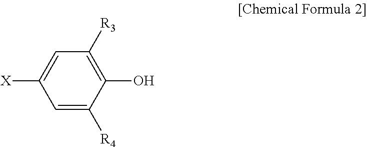

wherein, R₃ and R₄, which may the same or different, are independently hydroxyl or hydrogen, and X is a carboxyl group or an amine group.

For example, the polymer of Chemical Formula 1 can be prepared as illustrated in Reaction Schemes 1 to 5. In the reaction schemes, EDC stands for 1-ethyl-3-(3-dimethylaminopropyl)-carbodiimide, NHS for N-hydroxysuccinimide, TEA for triethylamine, DMAP for dimethylammonium pyridine, and NPCF for p-nitrophenylchloroformate.

In detail, the preparation of the polymer of Chemical Formula 1 may be achieved by (i) preparing a water-soluble polymer having amine, hydroxyl or carboxyl groups as a linker; (ii) bonding either a phenol derivative or an catechol derivative to the water-soluble polymer to form a conjugate of linker-phenol or aniline derivative; and (iii) grafting a polymer backbone having amine, carboxyl or hydroxyl groups with the conjugate.

Optionally, the step of adding a succinic anhydride or NPCF together with TEA and DMAP may be conducted between the steps (i) and (ii).

The presence of EDC and HNS may activate the phenol or catechol derivative to react with the water-soluble polymer. In addition, when the polymer backbone is grafted with the conjugate, EDC and HNS may be added to facilitate the reaction.

Before the step (iii), the conjugate may be further modified with a diamine compound.

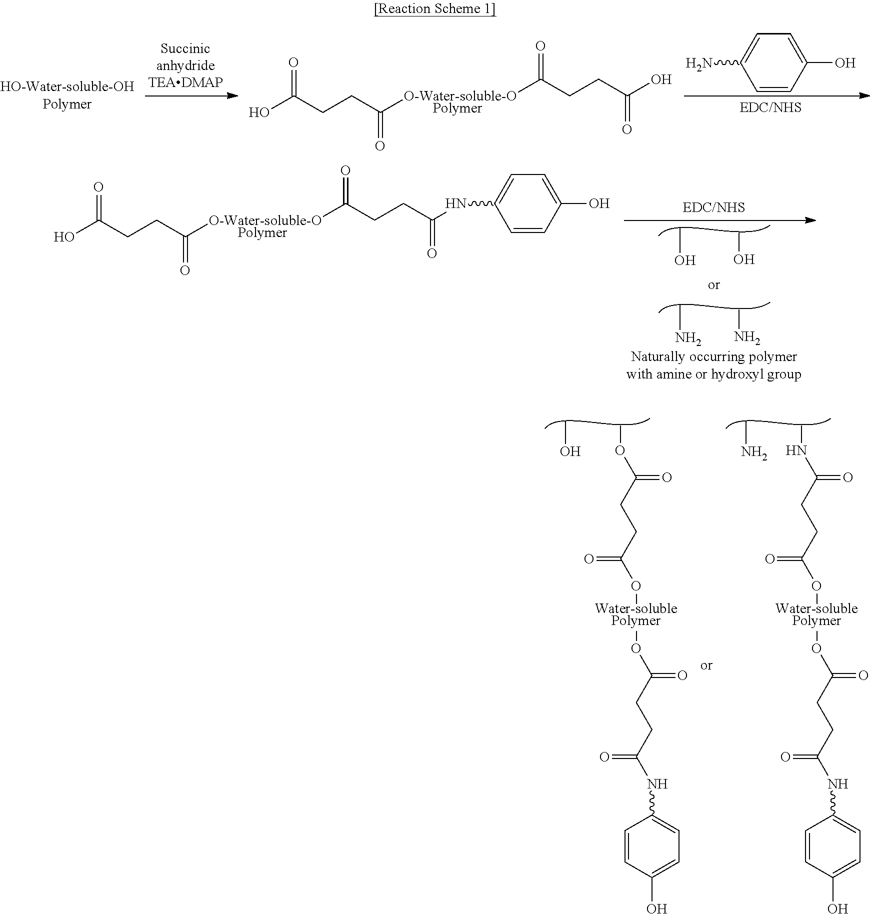

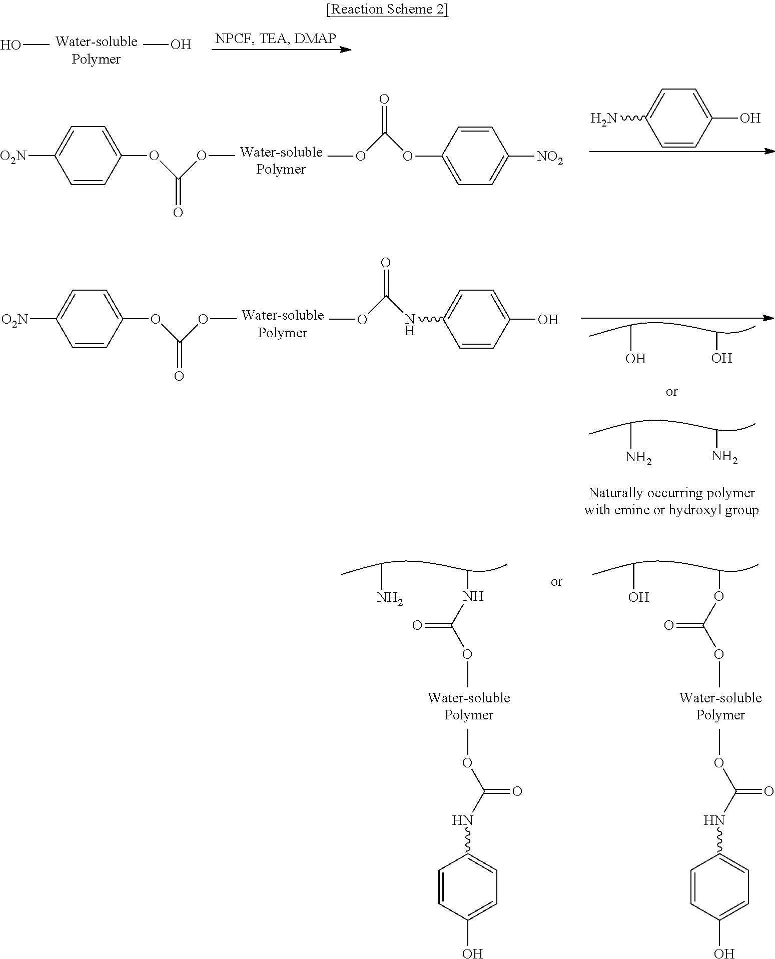

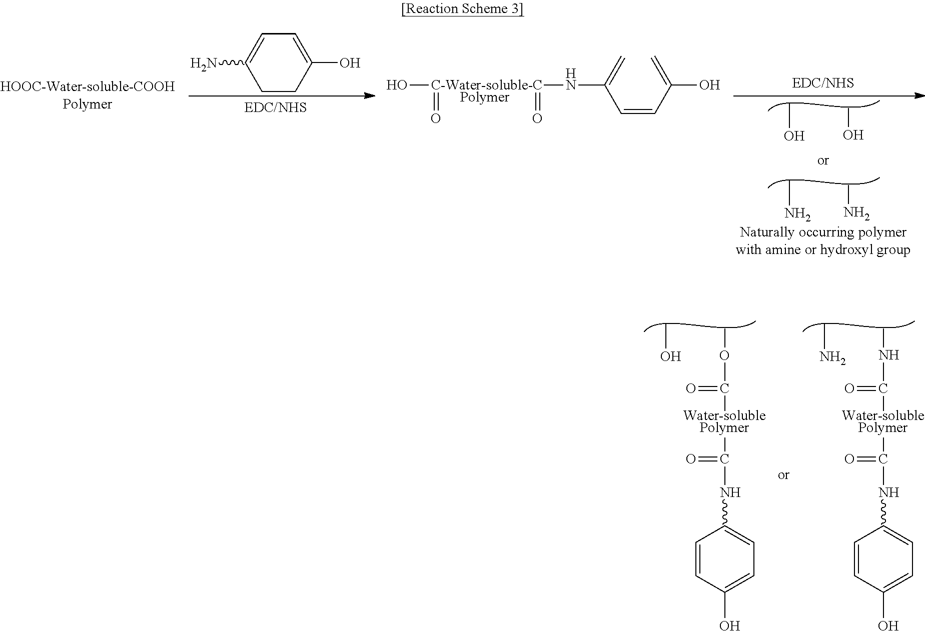

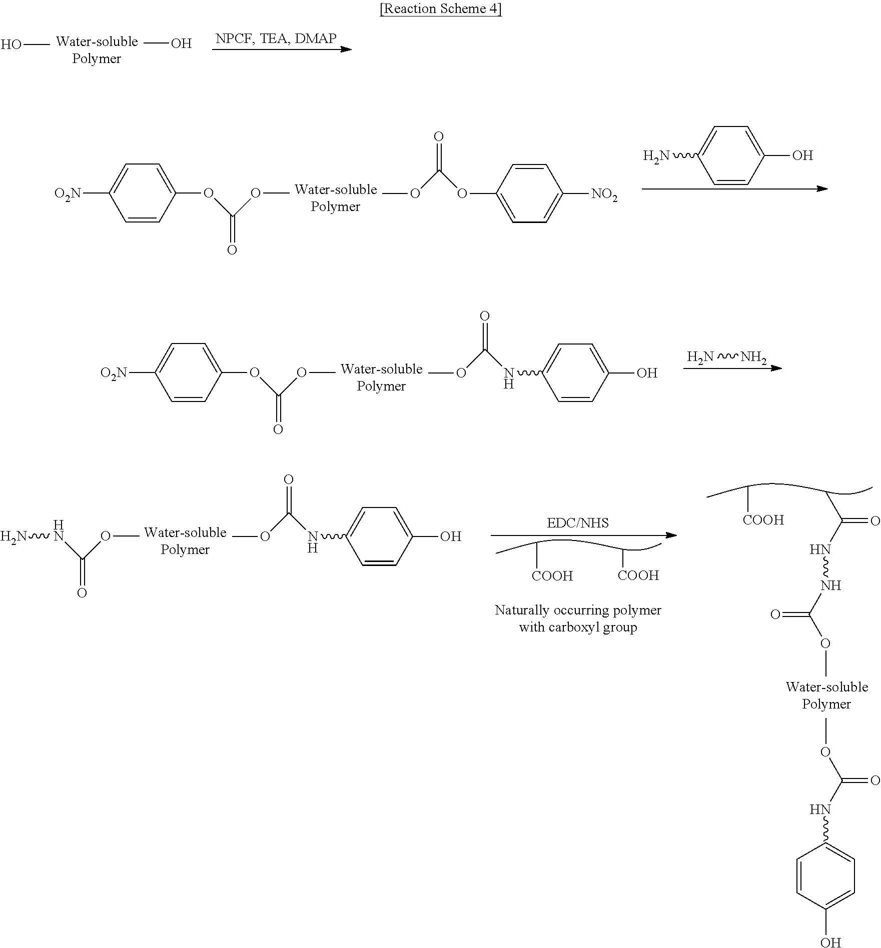

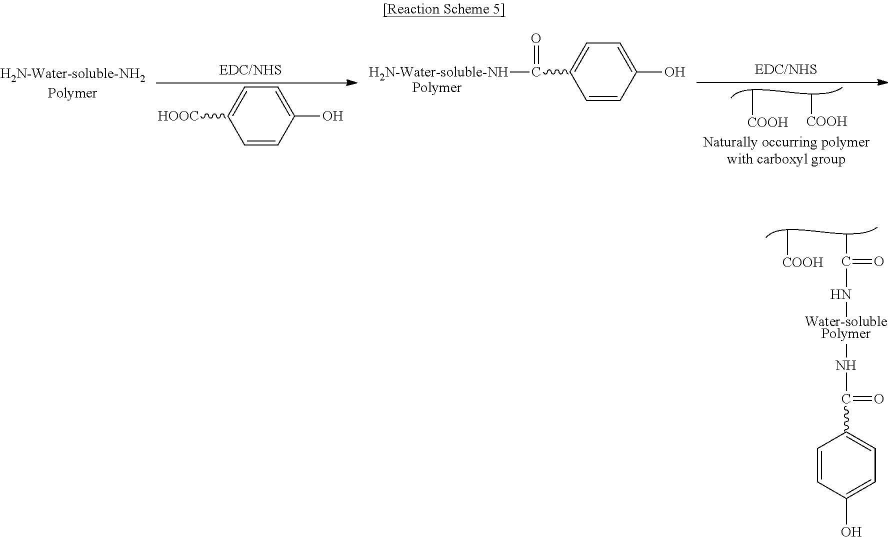

A polymer backbone suitable for use in the present invention may be derived from a polymer or one more selected from the group consisting of heparin, hyaluronic acid, collagen, gelatin, chitosan, cellulose, dextran, dextran sulfate, chondroitin sulfate, keratan sulfate, dermatan sulfate, alginate, albumin, fibronectin, laminin, elastin, vitronectin, fibrinogen, polyethylene glycol[PEG], polyethylene oxide[PEO], polyethylene imine[PEI], polypropylene oxide [PPO], polyvinyl alcohol [PVA], poly(N-isopropyl acrylamide) [polyNIPAM], polyfumarate, polyorganophosphagene, polyacrylic acid [polyAAc], polyacryl sulfonate, polyhydroxyethylmethacrylate [PolyHEMA], PEO-PPO-PEO (Pluronic® series), 4-arm PEO-PPO-PEO (Tetronic® series), PEG-PEI, PEG-PVA, PEG-PEI-PVA, PEI-PVA, poly(NIPAAM-co-AAc), poly(NIPAAM-co-HEMA), and combinations thereof, but are not limited thereto.

Also, the polymer backbone may be derived from a peptide, protein drug or one more and examples of the peptide or protein drug include fibroblast growth factor (FGF), vascular endothelial growth factor (VEGF), transforming growth factor (TGF), bone morphogenetic protein (BMP), human growth hormone (hGH), pig growth hormone (pGH), granulocyte colony-stimulating factor (G-CSF), erythropoietin (EPO), macrophage colony-stimulating factor (M-CSF), tumor necrosis factor (TNF), epidermal growth factor (EGF), platelet-derived growth factor (PDGF), interferon-α,β,γ, interleukin-2 (IL-2), calcitonin, nerve growth factor (NGF), growth hormone releasing hormone, angiotensin, luteinizing hormone releasing hormone (LHRH), luteinizing hormone releasing hormone agonist (LHRH agonist), insulin, thyrotropin-releasing hormone (TRH), angiostatin, endostatin, somatostatin, glucagon, endorphine, bacitracin, mergain, colistin, monoclonal antibodies, and vaccines, but are not limited thereto.

Also, in a preferred embodiment, the polymer backbone may be derived from anti-proliferative agents, anti-inflammatory agents, anti-thromobotic agents or combinations thereof. Examples of the anti-proliferative agents useful in the present invention include sirolimus (rapamycin), everolimus, pimecrolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenphosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, smc proliferation inhibitor-2w, epothilone A and B, mitoxantrone, azathioprine, mycophenolatmofetil, c-myc-antisense, b-myc-antisense, betulinic acid, camptothecin, PI-88 (sulfated oligosaccharide), melanocyte stimulating hormone(α-MSH), activated protein C, IL-1β inhibitors, thymosine α-1, fumaric acid and its esters, calcipotriol, tacalcitol, lapachol, β-lapachone, podophyllotoxin, betulin, podophyllic acid 2-ethylhydrazide, molgramostim (rhuGM-CSF), peginterferon α-2b, lenograstimn (r-HuG-CSF), filgrastim, macrogol, dacarbazine, basiliximab, daclizumab, selectin (cytokine antagonist), CETP inhibitors, cadherines, cytokinin inhibitors, COX-2 inhibitors, NFkB, angiopeptin, ciprofloxacin, fluoroblastin, monoclonal antibodies inhibitive of mycocyte proliferation, bFGF antagonists, probucol, prostaglandin, 1,11-dimethoxycanthin-6-one, 1-hydroxy-11-methoxycanthin-6-one, scopoletin, colchicine, NO donors such as pentaerythritol tetranitrate and syndnoeimine, S-nitroso derivatives, tamoxifen, staurosporine, β-estradiol, α-estradiol, estriol, estrone, ethinylestradiol, fosfestrol, medroxyprogesterone, estradiol cypionate, estradiol benzoate, tranilast, cancer therapeutic kamebakaurin and other terpenoids, verapamil, tyrosine kinase inhibitors (tyrphostines), cyclosporine A, paclitaxel and its derivatives such as 6-α-hydroxy-paclitaxel, baccatin, taxotere, macrocyclic oligomers (MCS) of natural or synthetic carbon suboxide and their derivatives, mofebutazone, acemetacin, diclofenac, lonazolac, dapsone, O-carbamoylphenoxyacetic acid, lidocaine, ketoprofen, mefenamicacid, piroxicam, meloxicam, chloroquine phosphate, penicillamine, tumstatin, avastin, D-24851, SC-58125, hydroxychloroquine, auranofin, sodium aurothiomalate, oxaceprol, celecoxib, β-sitosterin, ademetionine, myrtecaine, polidocanol, nonivamide, levomenthol, benzocaine, aescin, ellipticine, D-24851 Calbiochem, colcemid, cytochalasin A-E, indanocine, nocodazole, S 100 protein, bacitracin, vitronectin receptor antagonist, azelastine, guanidyl cyclase stimulator, tissue inhibitor of metal proteinase-1 and -2, free nucleic acids, nucleic acids, DNA and RNA fragments integrated into viral vectors, plasminogen activator inhibitor-1, plasminogen activator inhibitor-2, antisense oligonucleotides, VEGF inhibitors and IGF-1; examples of the anti-inflammatory agents useful in the present invention include natural or synthetic steroids such as cefadroxil, cefazolin, cefaclor, cefotaxim, tobramycin, gentamycin, dicloxacillin, oxacillin, leflunomide, anakinra, etanercept, sulfasalazine, etoposide, dicloxacillin, tetracycline, triamcinolone, mutamycin, procainamid, D24851, SC-58125, retinoic acid, quinidine, disopyramide, flecamide, propafenone, sotalol, amidorone, bryophyllin A, inotodiol, maquiroside A, ghalakinoside, mansonine, strebloside, hydrocortisone, betamethasone and dexamethasone, non-steroidal substances (NSAIDS) such as fenoprofen, ibuprofen, indomethacin, naproxen and phenylbutazone, antiprozoal agents such as acyclovir, ganciclovir, zidovudine, clotrimazole, flucytosine, griseofulvin, ketoconazole, miconazole, nystatin, terbinafine, chloroquine, mefloquine and quinine, hippocaesculin, barringtogenol-C21-angelate, 14-dehydroagrostistachin, agroskerin, agrostistachin, 17-hydroxyagrostistachin, ovatodiolid, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-β-hydroxypregnadiene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-AII, bisparthenolidine, periplocoside A, ghalakinoside, ursolic acid, deoxypsorospermin, sychorubin, ricin A, sanguinarine, manwu wheat acid, methylsorbifolin, sphatheliachromen, stizophyllin, strebloside, akagerine, dihydrousambarensine, hydroxyusambarine, strychnopentamine, strychnophylline, usambarine, usambarensine, daphnoretin, lariciresinol, methoxylariciresinol, syringaresinol, umbelliferon, afromoson, acetylvismione B, desacetylvismione A, vismione A and B, sulfur-containing amino acids such as cysteine, salts of the above-illustrated agents, and combination thereof; examples of the anti-thrombotic agents useful in the present invention include sulfone amide, metronidazol, argatroban, aspirin, abciximab, syntheticantithrombin, bivalirudin, coumadin, enoxaparin, antithrombotics such as desulphated and N-reacetylated heparin, tissue plasminogen activator, GpIIb/IIIa platelet membrane receptor, antibodies to factor Xa inhibitor, heparin, hirudin, r-hirudin, PPACK, protamin, sodium 2-methylthiazolidine-2,4-dicarboxylate, prourokinase, streptokinase, warfarin, urokinase, dipyramidole, trapidil, nitroprusside, PDGF antagonists such as triazolopyrimidine andseramin, captopril, cilazapril, lisinopril, enalapril, losartan, thioprotease inhibitors, prostacyclin, vapiprost, α-, β- and γ-interferon, histamine antagonist, serotonin blockers, apoptosis inhibitors, p65, NF-kB or Bcl-xL antisense oligonucleotides, halofuginone, nifedipine, tocopherol, vitamin B1, B2, B6 and B12, folic acid, tranilast, molsidomine, tea polyphenol, epicatechin gallate, epigallocatechingallate, Boswellinic acid and derivatives thereof, but are not limited thereto.

The phenol derivative useful in the present invention is selected from the group consisting of tyramine, hydroxyphenylacetic acid, hydroxypropionic acid, derivatives thereof, and a combination thereof. One or more compounds selected from the group consisting of L-dihydroxy phenylalanine (L-DOPA), dopamine, norepinephrine, epinephrine, epigallocatechin gallate, and derivatives thereof may be used as the catechol derivatives useful in the present invention.

As for the polymer used as a linker in the present invention, it may be selected from the group consisting of polyesters, polyanhydrides, polyorthoesters, polyurethanes, polyamides, polypeptides, polynuclear aliphatic hydrocarbons, polynuclear aromatic hydrocarbons, alkyl chains and a combination thereof.

Alternatively, the linker may be a hydrophilic polymer selected from the group consisting of polyethylene glycol-polylactic acid [PEG-PLA], polyethylene glycolpolycaprolactone [PEG-PCL], polyethylene glycol-poly(DL-lactic-co-glycolic acid)[PEG-PLGA], poly((propylene)fumarate), poly((ethylene)fumarate) and a combination thereof.

In another embodiment, the linker may be a hydrophilic polymer consisting of the group consisting of polyethylene glycol[PEG], polyethylene oxide[PEO], polyethylene imine[PEI], polypropylene oxide [PPO], polyvinyl alcohol [PVA], poly(N-isopropyl acrylamide) [polyNIPAM], polyfumarate, polyorganophosphagene, polyacrylic acid [polyAAc], polyacryl sulfonate, polyhydroxyethylmethacrylate [PolyHEMA] and a copolymer thereof. Examples of the copolymer include PEO-PPO-PEO (Pluronic® series), 4-arm PEO-PPO-PEO (Tetronic® series), PEG-PEI, PEG-PVA, PEG-PEI-PVA, PEI-PVA, poly(NIPAAM-co-AAc), poly(NIPAAM-co-HEMA), and combinations thereof, but are not limited thereto.

The polyphenol oxidase may be selected from among tyrosinase, catechol oxidase and a combination thereof, but is not limited thereto.

The substrate may be metallic or polymeric, and particularly may be a medical metal or polymer, but is not limited thereto.

For treating the substrate surface with a polyphenol oxidase solution, a method may be selected from the group consisting of spraying, injecting, painting, immersing, role coating and flow coating.

In accordance with the present invention, the immobilization level of the bioactive molecule can be adjusted with various factors including the concentration of PPO, reaction temperature and time, the initial concentration of the bioactive molecule including peptides, protein drugs or polymers, and the molecular weight of a linker between the phenol or catechol derivative and the polymer backbone.

When the substrate surface is treated therewith, the solution preferably contains the bioactive material at a concentration of from 0.001 to 50 wt % and PPO at a concentration of from 0.001 to 1 KU/ml. If the concentration of the bioactive molecule or PPO is outside the range, the immobilization may be performed ineffectively.

Effective immobilization may be achieved by treatment only within five minutes of treatment as well as for a longer period of time.

In an embodiment, a cell adhesion peptide containing a tyrosine residue (SEQ ID NO. 15: GRGDGGGGGY) may be immobilized to a metallic or polymeric surface in such a manner that the phenol group of the tyrosine is in situ converted to quinone/dopaquinone which is anchored to the surface via a coordinate bond.

In an embodiment of the present invention, naturally occurring polymers such as heparin, useful as an antithrombotic agent, gelatin, chitosan, etc., may provide the polymer backbone while a water-soluble polymer such as PEG may be used as a linker to which a phenol or catechol molecule is adapted so as to synthesize heparin-PEG-tyramine(HPT), gelatin-PEG-tyramine(GPT), mPEG-tyramine (mPTA) or chitosan-PEG-tyramine (CPT). In the presence of tyrosinase, the phenol or catechol group may be in situ converted into dopa/dopaquinone which can form a coordinate bond with a metal substrate or a polymer substrate, so that the bioactive molecule can be simply immobilized to the metal or polymer surface. In this context, the immobilization level of the bioactive molecule can be determined depending on various factors including the concentration of tyrosinase, reaction temperature and time, the amount of dissolved oxygen in the reaction solution, and the molecular weight of a linker between the phenol or catechol molecule and the polymer backbone.

In accordance with another aspect thereof, the present invention provides a method for immobilizing a bioactive molecule using polyphenol oxidase, comprising preparing a polymer having tyramine introduced to both ends thereof (step 1); treating a substrate surface with the polymer together with polyphenol oxidase to convert the tyramine into a dopaquinone which forms a coordinate bond with the substrate surface, thus anchoring the polymer to the surface (step 2); and introducing a bioactive molecule into the polymer backbone through a Michael addition reaction or imine formation reaction with the dopaquinone molecule, the bioactive molecule being selected from the group consisting of an anti-proliferative agent, anti-inflammatory agent, an anti-thrombotic agent and a combination thereof (step 3).

The polyphenol oxidase, the substrate and the treatment method are as described above.

In an embodiment of the present invention, tyramine-poly(ethylene glycol)-tyramine(PEG-TA) is anchored to a metal or polymer surface using tyrosinase, and a bioactive molecule can be introduced into the polymer backbone through a Michael addition reaction or imine formation reaction with the dopaquinone molecule formed by the enzymatic action and thus can be immobilized to the surface. Thus, a bioactive molecule, for example, a growth factor, can be immobilized to a solid phase, for example, a metal or polymer surface.

Based on the surface immobilization of bioactive molecules using polyphenol oxidase in accordance with the present invention, various bioactive molecules such as osteogenetic peptides and growth factors can be simply immobilized to medical metal or polymer substrate surfaces. For example, cell adhesion peptides may be immobilized to orthopedic or dental implants which can be then effectively used to induce rapid osteogenesis after being transplanted. In another embodiment, antithrombotic agents and/or entothelialization-inducing agents may be immobilized to medical substrates for vascular systems, such as stents and artificial blood vessels, thus guaranteeing hemocompatibility to the medical substrates.

### Advantageous Effects of Invention

As described hitherto, the method for immobilizing bioactive materials to a solid phase using polyphenol oxidase, especially tyrosinase, finds various applications in the medical engineering field. For example, cell adhesion peptides can be readily immobilized to an orthopedic or dental substrate which is then transplanted with the concomitant induction of osteogenesis. When applied to medical substrates for vascular systems, such as stents and artificial blood vessels, the method of the present invention allows the immobilization of antithrombotic agents and thus guarantees hemocompatibility to the medical substrates.

### BRIEF DESCRIPTION OF DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 is a schematic diagram showing a procedure of immobilizing a bioactive molecule on a substrate surface,

FIG. 2 is a reaction scheme showing the synthesis of a heparin-poly(ethylene glycol)-tyramine (HPT) copolymer,

FIG. 3 is a reaction scheme showing the synthesis of a gelatin-poly(ethylene glycol)-tyramine (GPT) copolymer,

FIG. 4 is a reaction scheme showing the synthesis of tyramine-poly(ethylene glycol)-tyramine (PEG-TA),

FIG. 5 is a reaction schem showing the synthesis of a methoxy poly(ethylene glycol)-tyramine (mPTA),

FIG. 6 is of saturation curves for tyramine showing the relationship between time and conversion ratio,

FIG. 7 is a graph showing contact angles on various metal specimens including a dopamine-immobilized metal specimen and a metal specimen on which tyramine was immobilized in the presence of tyrosinase,

FIG. 8 is a graph showing amine distributions over the metal specimens,

FIG. 9 is a schematic illustration showing the immobilization of RGD, gelatin or heparin on a metal surface by simple immersion,

FIG. 10 is a schematic illustration showing the introduction of a protein drug (e.g. growth factor) onto a metal or polymer substrate by coupling with dopaquinone molecules which are formed when a tyramine-poly(ethylene glycol)-tyramine (PEG-TA) is anchored to the surface of the metal or polymer substrate in the presence of tyrosinase by a simple immersion procedure.

FIG. 11 is a graph showing concentrations of the growth factor immobilized on metal surfaces using tyrosinase.

FIG. 12 is a schematic illustration showing the immobilization of RGD and/or YIGSR on a polymer (polyurethane) surface by simple immersion,

FIG. 13 is a schematic illustration showing the immobilization of mPTA on a metal, ceramic or polymer substrate by simply immersion,

FIG. 14 shows contact angles on the surfaces of mPTA-immobilized substrates,

FIG. 15 shows contact angles on metal surfaces on which biomolecules are immobilized using tyrosinase.

FIG. 16 is of bar graphs showing changes in the RGD concentration immobilized on a surface with the concentrations of tyrosinase and initially used RGD and the reaction time.

FIG. 17 is of bar graphs showing changes in the heparin concentration immobilized on a surface with the concentrations of tyrosinase and initially used HPT and the reaction time.

FIG. 18 is of bar graphs showing changes in the gelatin concentration immobilized on a surface with the concentrations of tyrosinase and initially used GPT and the reaction time.

FIG. 19 shows surface concentrations of RGD and YIGSR introduced using tyrosinase.

FIG. 20 is a graph showing results of assacy for the stability of RGD, gelatin and heparin, immobilized on metal surfaces using tyrosinase.

FIG. 21 is of bar graphs showing results of the assay for cell attachment ability of stainless steel and titanium surfaces on which the cell adhesion peptides RGD and gelatin are immobilized,

FIG. 22 shows morphologies of the cells cultured on the titanium surface to which the cell adhesion peptide RGD is immobilized,

FIG. 23 is a graph showing cell proliferation ability of the stainless steel surfaces on which RGD or gelatin is immobilized with the aid of tyrosinase,

FIG. 24 is a graph showing the activity of heparin immobilized on a surface with the aid of tyrosinase,

FIG. 25 is a schematic illustration showing the preparation of artificial blood vessels made of polyurethane by electrospinning, together with electron microphotographs showing structures of the artificial blood vessels,

FIG. 26 is of photographs showing an animal experiment using a bioactive molecule (heparin, RGD or YIGSR)-immobilized artificial blood vessel made of polyurethane (carotid artery before surgery (a), carotid artery cut after clamping (b), artificial blood vessel ligated to the artery at the resected site (c), artificial blood vessel implanted (d)).

FIG. 27 is of photographs of the artificial blood vessels excised after implantation, showing no occurrence of stenosis (PU: control, Pep: PU-PEG-Heparin/YIGSR/RGD); and

FIG. 28 is of photographs of the artificial blood vessels excised after implantation, showing histological compatibility thereof (PU (a), PU-PEG-Heparin/YIGSR/RGD (b), PU-PEG-Heparin(c)).

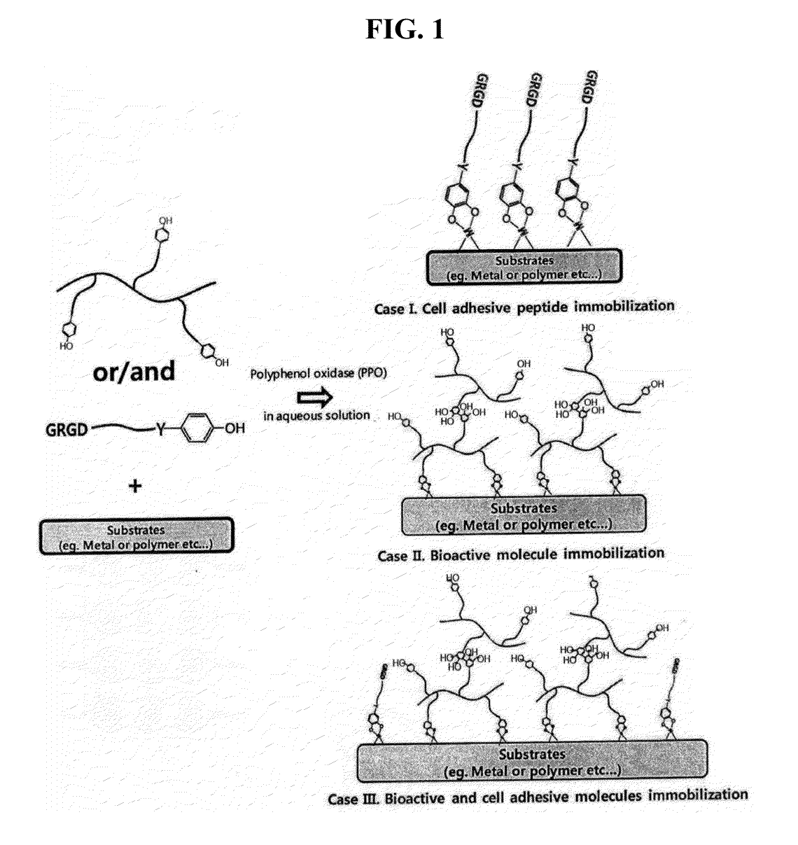

### BEST MODE FOR CARRYING OUT THE INVENTION

The present invention provides a method for immobilization of a bioactive molecule on a surface, comprising: preparing a phenol-, catechol- or its derivative containing bioactive molecule (step 1); and treating a substrate surface with the bioactive molecule in presence of polyphenol oxidase to immobilize the bioactive molecule onto the surface (step 2).

The bioactive molecule contains a cell adhesion peptide containing a tyrosine. Preferable examples of the cell adhesion peptide include peptides of SEQ ID NO. 1 (RGD-Y), SEQ ID NO. 2 (KQAGDV-Y), SEQ ID NO. 3 (YIGSR), SEQ ID NO. 4 (REDV-Y), SEQ ID NO. 5 (IKVAN-Y), SEQ ID NO. 6 (RNIAEIIKDI-Y), SEQ ID NO. 7 (KHIFSDDSSE-Y), SEQ ID NO. 8 (VPGIG-Y), SEQ ID NO. 9 (FHRRIKA-Y), SEQ ID NO. 10 (KRSR-Y), SEQ ID NO. 11 (NSPVNSKIPKACCVPTELSAI-Y), SEQ ID NO. 12 (APGL-Y), SEQ ID NO. 13 (VRN-Y) and SEQ ID NO. 14 (AAAAAAAAA-Y) and a combination thereof.

The bioactive molecule comprises one or more polymers, represented by the following Chemical Formula 1, in which a phenol or catechol derivative is grafted via a linker or directly to a polymer backbone:

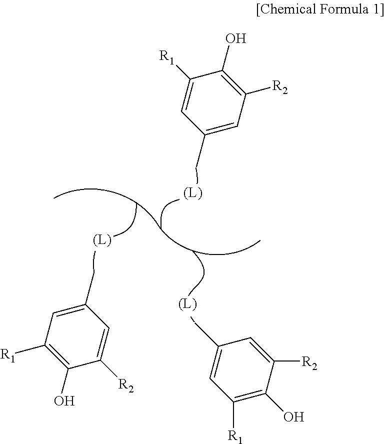

wherein, R₁ and R₂, which maythe same or different, are independently hydroxyl or

Hydrogen; and L is a polymeric linker.

The biomolecule is prepared by grafting a phenol or catechol derivative represented by the following Chemical Formula 2 to the polymer backbone having amino, hydroxyl or carboxyl groups through an amide, urethane, urea or ester bond, with a water-soluble polymer serving as the linker:

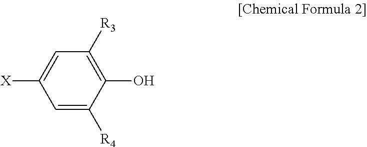

wherein, R₃ and R₄, which may the same or different, are independently hydroxyl or hydrogen, and X is a carboxyl group or an amine group.

The polymer backbone is derived from a polymer or one more selected from the group consisting of heparin, hyaluronic acid, collagen, gelatin, chitosan, cellulose, dextran, dextran sulfate, chondroitin sulfate, keratan sulfate, dermatan sulfate, alginate, albumin, fibronectin, laminin, elastin, vitronectin, fibrinogen, polyethylene glycol[PEG], polyethylene oxide[PEO], polyethylene imine[PEI], polypropylene oxide [PPO], polyvinyl alcohol [PVA], poly(N-isopropyl acrylamide) [polyNIPAM], polyfumarate, polyorganophosphagene, polyacrylic acid [polyAAc], polyacryl sulfonate, polyhydroxyethylmethacrylate [PolyHEMA], PEO-PPO-PEO (Pluronic® series), 4-arm PEO-PPO-PEO (Tetronic® series), PEG-PEI, PEG-PVA, PEG-PEI-PVA, PEI-PVA, poly(NIPAAM-co-AAc), poly (NIPAAM-co-HEMA), and combinations thereof.

The polymer backbone may be derived from a polymer or one more selected from the group consisting of fibroblast growth factor (FGF), vascular endothelial growth factor (VEGF), transforming growth factor (TGF), bone morphogenetic protein (BMP), human growth hormone (hGH), pig growth hormone (pGH), granulocyte colony-stimulating factor (G-CSF), erythropoietin (EPO), macrophage colony-stimulating factor (M-CSF), tumor necrosis factor (TNF), epidermal growth factor (EGF), platelet-derived growth factor (PDGF), interferon-α,β,γ, interleukin-2 (IL-2), calcitonin, nerve growth factor (NGF), growth hormone releasing hormone, angiotensin, luteinizing hormone releasing hormone (LHRH), luteinizing hormone releasing hormone agonist (LHRH agonist), insulin, thyrotropin-releasing hormone (TRH), angiostatin, endostatin, somatostatin, glucagon, endorphine, bacitracin, mergain, colistin, monoclonal antibodies, and vaccines.

The polymer backbone is derived from the group consisting of an anti-proliferative agent, an anti-inflammatory agent, an anti-thromobotic agent and a combination thereof, said anti-proliferative agent being selected from among sirolimus (rapamycin), everolimus, pimecrolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenphosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, smc proliferation inhibitor-2w, epothilone A and B, mitoxantrone, azathioprine, mycophenolatmofetil, c-myc-antisense, b-myc-antisense, betulinic acid, camptothecin, PI-88 (sulfated oligosaccharide), melanocyte stimulating hormone(α-MSH), activated protein C, IL-1β inhibitors, thymosine α-1, fumaric acid and its esters, calcipotriol, tacalcitol, lapachol, β-lapachone, podophyllotoxin, betulin, podophyllic acid 2-ethylhydrazide, molgramostim (rhuGM-CSF), peginterferon α-2b, lenograstimn (r-HuG-CSF), filgrastim, macrogol, dacarbazine, basiliximab, daclizumab, selectin (cytokine antagonist), CETP inhibitors, cadherines, cytokinin inhibitors, COX-2 inhibitors, NFkB, angiopeptin, ciprofloxacin, fluoroblastin, monoclonal antibodies inhibitive of mycocyte proliferation, bFGF antagonists, probucol, prostaglandin, 1,11-dimethoxycanthin-6-one, 1-hydroxy-11-methoxycanthin-6-one, scopoletin, colchicine, NO donors including pentaerythritol tetranitrate or syndnoeimine, S-nitroso derivatives, tamoxifen, staurosporine, β-estradiol, α-estradiol, estriol, estrone, ethinylestradiol, fosfestrol, medroxyprogesterone, estradiol cypionate, estradiol benzoate, tranilast, cancer therapeutic kamebakaurin and other terpenoids, verapamil, tyrosine kinase inhibitors (tyrphostines), cyclosporine A, paclitaxel and its derivatives such as 6-α-hydroxy-paclitaxel, baccatin, taxotere, macrocyclic oligomers (MCS) of natural or synthetic carbon suboxide and their derivatives, mofebutazone, acemetacin, diclofenac, lonazolac, dapsone, O-carbamoylphenoxyacetic acid, lidocaine, ketoprofen, mefenamicacid, piroxicam, meloxicam, chloroquine phosphate, penicillamine, tumstatin, avastin, D-24851, SC-58125, hydroxychloroquine, auranofin, sodium aurothiomalate, oxaceprol, celecoxib, β-sitosterin, ademetionine, myrtecaine, polidocanol, nonivamide, levomenthol, benzocaine, aescin, ellipticine, D-24851 Calbiochem, colcemid, cytochalasin A-E, indanocine, nocodazole, S 100 protein, bacitracin, vitronectin receptor antagonist, azelastine, guanidyl cyclase stimulator, tissue inhibitor of metal proteinase-1 and -2, free nucleic acids, nucleic acids, DNA and RNA fragments integrated into viral vectors, plasminogen activator inhibitor-1, plasminogen activator inhibitor-2, antisense oligonucleotides, VEGF inhibitors and IGF-1; said anti-inflammatory agent being selected from among natural or synthetic steroids including cefadroxil, cefazolin, cefaclor, cefotaxim, tobramycin, gentamycin, dicloxacillin, oxacillin, leflunomide, anakinra, etanercept, sulfasalazine, etoposide, dicloxacillin, tetracycline, triamcinolone, mutamycin, procainamid, D24851, SC-58125, retinoic acid, quinidine, disopyramide, flecamide, propafenone, sotalol, amidorone, bryophyllin A, inotodiol, maquiroside A, ghalakinoside, mansonine, strebloside, hydrocortisone, betamethasone and dexamethasone, non-steroidal substances (NSAIDS) including fenoprofen, ibuprofen, indomethacin, naproxen and phenylbutazone, antiprozoal agents such as acyclovir, ganciclovir, zidovudine, clotrimazole, flucytosine, griseofulvin, ketoconazole, miconazole, nystatin, terbinafine, chloroquine, mefloquine and quinine, hippocaesculin, barringtogenol-C21-angelate, 14-dehydroagrostistachin, agroskerin, agrostistachin, 17-hydroxyagrostistachin, ovatodiolid, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-β-hydroxypregnadiene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-AII, bisparthenolidine, periplocoside A, ghalakinoside, ursolic acid, deoxypsorospermin, sychorubin, ricin A, sanguinarine, manwu wheat acid, methylsorbifolin, sphatheliachromen, stizophyllin, strebloside, akagerine, dihydrousambarensine, hydroxyusambarine, strychnopentamine, strychnophylline, usambarine, usambarensine, daphnoretin, lariciresinol, methoxylariciresinol, syringaresinol, umbelliferon, afromoson, acetylvismione B, desacetylvismione A, vismione A and B, sulfur-containing amino acids including cysteine, salts of the above-illustrated agents, and a combination thereof; the anti-thrombotic agent is selected from among sulfone amide, metronidazol, argatroban, aspirin, abciximab, syntheticantithrombin, bivalirudin, coumadin, enoxaparin, antithrombotics including desulphated and N-reacetylated heparin, tissue plasminogen activator, GpIIb/IIIa platelet membrane receptor, antibodies to factor Xa inhibitor, heparin, hirudin, r-hirudin, PPACK, protamin, sodium 2-methylthiazolidine-2,4-dicarboxylate, prourokinase, streptokinase, warfarin, urokinase, dipyramidole, trapidil, nitroprusside, PDGF antagonists including triazolopyrimidine andseramin, captopril, cilazapril, lisinopril, enalapril, losartan, thioprotease inhibitors, prostacyclin, vapiprost, α-, β- and γ-interferon, histamine antagonist, serotonin blockers, apoptosis inhibitors, p65, NF-kB or Bcl-xL antisense oligonucleotides, halofuginone, nifedipine, tocopherol, vitamin B1, B2, B6 and B12, folic acid, tranilast, molsidomine, tea polyphenol, epicatechin gallate, epigallocatechingallate, Boswellinic acid and derivatives thereof, and a combination thereof.

The phenol derivative useful in the present invention is selected from the group consisting of tyramine, hydroxyphenylacetic acid, hydroxypropionic acid, derivatives thereof, and a combination thereof.

The catechol derivative is selected from the group consisting of L-dihydroxy phenylalanine (L-DOPA), dopamine, norepinephrine, epinephrine, epigallocatechin gallate, and derivatives thereof, and a combination thereof.

The linker is a hydrophilic polymer selected from the group consisting of polyesters, polyanhydrides, polyorthoesters, polyurethanes, polyamides, polypeptides, polynuclear aliphatic hydrocarbons, polynuclear aromatic hydrocarbons, alkyl chains and a combination thereof.

The linker is a hydrophilic polymer selected from the group consisting of polyethylene glycol-polylactic acid [PEG-PLA], polyethylene glycol-polycaprolactone [PEG-PCL], polyethylene glycol-poly(DL-lactic-co-glycolic acid)[PEG-PLGA], poly((propylene)fumarate), poly((ethylene)fumarate) and a combination thereof.

The linker is a hydrophilic polymer selected from the group consisting of polyethylene glycol[PEG], polyethylene oxide[PEO], polyethylene imine[PEI], polypropylene oxide [PPO], polyvinyl alcohol [PVA], poly(N-isopropylacrylamide)[polyNIPAM], polyfumarate, polyorgano phosphagene, polyacrylic acid [polyAAc], polyacryl sulfonate, polyhydroxyethylmethacrylate [PolyHEMA] and a copolymer thereof.

The copolymer is selected from the group consisting of PEO-PPO-PEO (Pluronic® series), 4-armPEO-PPO-PEO(Tetronic® series), PEG-PEI, PEG-PVA, PEG-PEI-PVA, PEI-PVA, poly(NIPAAM-co-AAc), poly(NIPAAM-co-HEMA), and a combination thereof.

The polyphenol oxidase is selected from the group consisting of tyrosinase, catechol oxidase and a combination thereof.

The substrate is a metal or a polymer.

The substrate surface is treated with a solution containing the bioactive molecule and polyphenol oxidase using a method selected from the group consisting of spraying, injecting, painting, immersing, role coating and flow coating.

The solution contains the bioactive material at a concentration of from 0.001 to 50 wt % and PPO at a concentration of from 0.001 to 1 KU/ml.

The substrate surface is treated for 5 min to 1 hr.

The present invention provides a method for immobilizing a bioactive molecule using polyphenol oxidase, comprising: preparing a polymer having tyramine introduced to both ends thereof (step 1); treating a substrate surface with the polymer together with polyphenol oxidase to convert the tyramine into a dopaquinone which forms a coordinate bond with the substrate surface, thus anchoring the polymer to the surface (step 2); and introducing a bioactive molecule into the polymer backbone through a Michael addition reaction or imine formation reaction with the dopaquinone molecule, the bioactive molecule being selected from the group consisting of an anti-proliferative agent, anti-inflammatory agent, an anti-thrombotic agent and a combination thereof (step 3).

The bioactive molecule is selected from the group consisting of fibroblast growth factor (FGF), vascular endothelial growth factor (VEGF), transforming growth factor (TGF), bone morphogenetic protein (BMP), human growth hormone (hGH), pig growth hormone (pGH), granulocyte colony-stimulating factor (G-CSF), erythropoietin (EPO), macrophage colony-stimulating factor (M-CSF), tumor necrosis factor (TNF), epidermal growth factor (EGF), platelet-derived growth factor (PDGF), interferon-α,β,γ, interleukin-2 (IL-2), calcitonin, nerve growth factor (NGF), growth hormone releasing hormone, angiotensin, luteinizing hormone releasing hormone (LHRH), luteinizing hormone releasing hormone agonist (LHRH agonist), insulin, thyrotropin-releasing hormone (TRH), angiostatin, endostatin, somatostatin, glucagon, endorphine, bacitracin, mergain, colistin, monoclonal antibodies, vaccines, and a combination thereof.

The anti-proliferative agent is selected from among sirolimus (rapamycin), everolimus, pimecrolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenphosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, smc proliferation inhibitor-2w, epothilone A and B, mitoxantrone, azathioprine, mycophenolatmofetil, c-myc-antisense, b-myc-antisense, betulinic acid, camptothecin, PI-88 (sulfated oligosaccharide), melanocyte stimulating hormone(α-MSH), activated protein C, IL-1β inhibitors, thymosine α-1, fumaric acid and its esters, calcipotriol, tacalcitol, lapachol, β-lapachone, podophyllotoxin, betulin, podophyllic acid 2-ethylhydrazide, molgramostim (rhuGM-CSF), peginterferon α-2b, lenograstimn (r-HuG-CSF), filgrastim, macrogol, dacarbazine, basiliximab, daclizumab, selectin (cytokine antagonist), CETP inhibitors, cadherines, cytokinin inhibitors, COX-2 inhibitors, NFkB, angiopeptin, ciprofloxacin, fluoroblastin, monoclonal antibodies inhibitive of mycocyte proliferation, bFGF antagonists, probucol, prostaglandin, 1,11-dimethoxycanthin-6-one, 1-hydroxy-11-methoxycanthin-6-one, scopoletin, colchicine, NO donors including pentaerythritol tetranitrate or syndnoeimine, S-nitroso derivatives, tamoxifen, staurosporine, β-estradiol, α-estradiol, estriol, estrone, ethinylestradiol, fosfestrol, medroxyprogesterone, estradiol cypionate, estradiol benzoate, tranilast, cancer therapeutic kamebakaurin and other terpenoids, verapamil, tyrosine kinase inhibitors (tyrphostines), cyclosporine A, paclitaxel and its derivatives such as 6-α-hydroxy-paclitaxel, baccatin, taxotere, macrocyclic oligomers (MCS) of natural or synthetic carbon suboxide and their derivatives, mofebutazone, acemetacin, diclofenac, lonazolac, dapsone, O-carbamoylphenoxyacetic acid, lidocaine, ketoprofen, mefenamicacid, piroxicam, meloxicam, chloroquine phosphate, penicillamine, tumstatin, avastin, D-24851, SC-58125, hydroxychloroquine, auranofin, sodium aurothiomalate, oxaceprol, celecoxib, β-sitosterin, ademetionine, myrtecaine, polidocanol, nonivamide, levomenthol, benzocaine, aescin, ellipticine, D-24851 Calbiochem, colcemid, cytochalasin A-E, indanocine, nocodazole, S 100 protein, bacitracin, vitronectin receptor antagonist, azelastine, guanidyl cyclase stimulator, tissue inhibitor of metal proteinase-1 and -2, free nucleic acids, nucleic acids, DNA and RNA fragments integrated into viral vectors, plasminogen activator inhibitor-1, plasminogen activator inhibitor-2, antisense oligonucleotides, VEGF inhibitors and IGF-1; the anti-inflammatory agent is selected from among natural or synthetic steroids including cefadroxil, cefazolin, cefaclor, cefotaxim, tobramycin, gentamycin, dicloxacillin, oxacillin, leflunomide, anakinra, etanercept, sulfasalazine, etoposide, dicloxacillin, tetracycline, triamcinolone, mutamycin, procainamid, D24851, SC-58125, retinoic acid, quinidine, disopyramide, flecamide, propafenone, sotalol, amidorone, bryophyllin A, inotodiol, maquiroside A, ghalakinoside, mansonine, strebloside, hydrocortisone, betamethasone and dexamethasone, non-steroidal substances (NSAIDS) including fenoprofen, ibuprofen, indomethacin, naproxen and phenylbutazone, antiprozoal agents such as acyclovir, ganciclovir, zidovudine, clotrimazole, flucytosine, griseofulvin, ketoconazole, miconazole, nystatin, terbinafine, chloroquine, mefloquine and quinine, hippocaesculin, barringtogenol-C21-angelate, 14-dehydroagrostistachin, agroskerin, agrostistachin, 17-hydroxyagrostistachin, ovatodiolid, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-β-hydroxypregnadiene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-AII, bisparthenolidine, periplocoside A, ghalakinoside, ursolic acid, deoxypsorospermin, sychorubin, ricin A, sanguinarine, manwu wheat acid, methylsorbifolin, sphatheliachromen, stizophyllin, strebloside, akagerine, dihydrousambarensine, hydroxyusambarine, strychnopentamine, strychnophylline, usambarine, usambarensine, daphnoretin, lariciresinol, methoxylariciresinol, syringaresinol, umbelliferon, afromoson, acetylvismione B, desacetylvismione A, vismione A and B, sulfur-containing amino acids including cysteine, salts of the above-illustrated agents, and a combination thereof; and the anti-thrombotic agent is selected from among sulfone amide, metronidazol, argatroban, aspirin, abciximab, syntheticantithrombin, bivalirudin, coumadin, enoxaparin, antithrombotics including desulphated and N-reacetylated heparin, tissue plasminogen activator, GpIIb/IIIa platelet membrane receptor, antibodies to factor Xa inhibitor, heparin, hirudin, r-hirudin, PPACK, protamin, sodium 2-methylthiazolidine-2,4-dicarboxylate, prourokinase, streptokinase, warfarin, urokinase, dipyramidole, trapidil, nitroprusside, PDGF antagonists including triazolopyrimidine andseramin, captopril, cilazapril, lisinopril, enalapril, losartan, thioprotease inhibitors, prostacyclin, vapiprost, α-, β- and γ-interferon, histamine antagonist, serotonin blockers, apoptosis inhibitors, p65, NF-kB or Bcl-xL antisense oligonucleotides, halofuginone, nifedipine, tocopherol, vitamin B1, B2, B6 and B12, folic acid, tranilast, molsidomine, tea polyphenol, epicatechin gallate, epigallocatechingallate, Boswellinic acid and derivatives thereof, and a combination thereof.

### MODE FOR THE INVENTION

In an embodiment of the present invention, a cell adhesion peptide (SEQ ID NO. 15: GRGDGGGGGY), heparin-poly(ethylene glycol)-tyramine (HPT), gelatin-poly(ethylene glycol)-tyramine(GPT), and methoxy polyethylene glycol-tyramine (mPTA) were immobilized onto respective metallic or polymeric surfaces by a simple immersion method using tyrosinase, and assayed for physicochemical properties of the surfaces, cell affinity, function, histocompatibility and hemocompatibility.

In addition, bioactive molecules and drugs, such as growth factors, were immobilized to a metallic or polymeric surface through a dopaquinone molecule formed after tyramine-poly(ethylene glycol)-tyramine (PEG-TA) was anchored onto the surface using tyrosinase, and assayed for their activities on the surface.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Synthesis Example 1

### Synthesis of Heparin-Poly(ethylene glycol)-Tyramine (HPT)

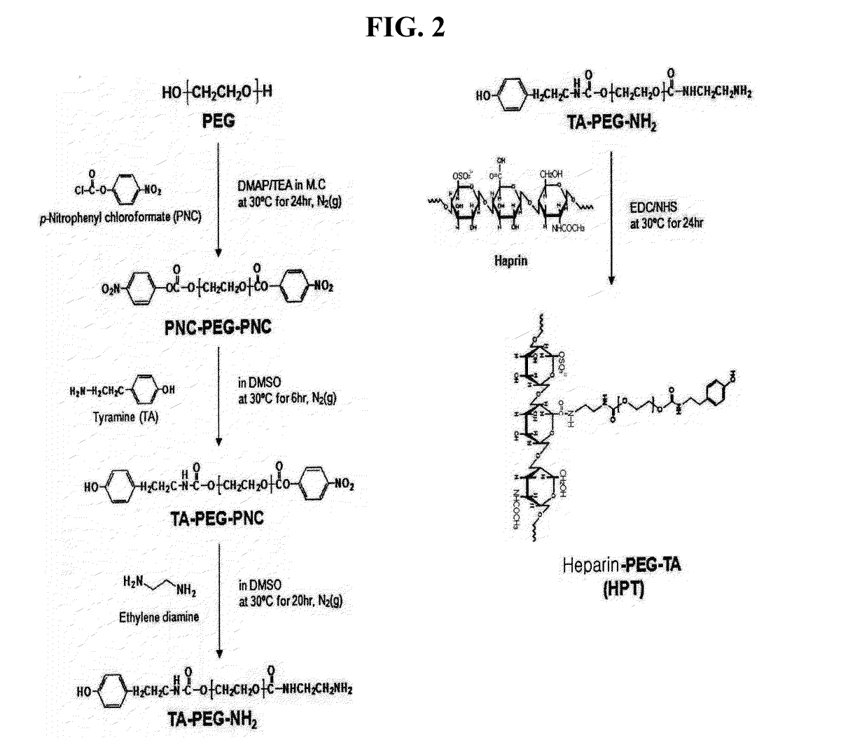

The synthesis of HPT is schematically illustrated in FIG. 2.

### 1. Synthesis of poly(ethylene glycol)-(p-nitrophenylchloroformate) [PEG-PNC]

A solution of 10 g (2.9 mmol) of PEG in 100 mL of MC was mixed sequentially with a solution of 0.779 g (6.38 mmol) of 4-dimethylaminopyridine (DMAP) and 0.645 g (6.38 mmol) of triethylamine (TEA) in 10 mL of MC and a solution of 1.286 g (6.38 mmol) of PNC in 50 mL of MC, with the molar ratio of PEG:DMAP:TEA:PNC being 1:2.2:2.2:2.2. A reaction was performed at 30° C. for 24 hrs in a nitrogen atmosphere.

After completion of the reaction, reagents which remained unreacted were removed by filtering the reaction mixture. This filtrate was then concentrated using a rotary evaporator. The concentrate was dropwise added to 1600 mL of chilled ether to form precipitates which were then obtained by filtration. The filtrate was allowed to stand for 24 hrs in a vacuum oven to remove the remaining organic solvents to afford the desired compound (PEG-PNC) as a white powder.

### 2. Synthesis of aminated poly(ethylene glycol)-tyramine (PTA)

To a solution of 5 g (1.25 mmol) of PEG-PNC was added 0.174 g (1.25 mmol) of tyramine (TA) in 100 mL of methylene chloride (MC), with the molar ratio of PEG-PNC:TA being 1:1. A reaction was performed at 30° C. for 6 hrs in a nitrogen atmosphere. Thereafter, a solution of 2.254 g (37.5 mmol) of ethylene diamine in 50 mL of MC was added, with the molar ratio of PEG-PNC:ethylene diamine being 1:30. They were reacted at 30° C. for 24 hrs.

After completion of the reaction, the reaction mixture was filtered to remove the remaining reagents, and concentrated using a rotary evaporator. The concentrate was dropwise added to 1600 mL of chilled ether to form precipitates which were then obtained by filtration. The filtrate was allowed to stand for 24 hrs in a vacuum oven to remove the remaining organic solvents to afford the desired compound (PTA) as a white powder.

### 3. Synthesis of heparin-poly(ethylene glycol)-tyramine(HPT)

0.065 g (0.334 mmol) of EDC and 0.019 g (0.167 mmol) of NHS were sequentially added at intervals of 15 min to a solution of 1 g of heparin in 30 mL of distilled water. In a reaction flask, the resulting solution was mixed with a solution of 1.420 g (0.334 mmol) of PTA in 10 mL of distilled water before a reaction was performed at 30° C. for 24 hrs.

After removal of the remaining reagents by filtration, the solution was subjected for 3˜4 days to membrane dialysis against distilled water (cutoff Mw 6000-8000 Da). The dialized solution was lyophilized to afford the desired compound (HPT) as a white powder.

### Synthesis Example 2

### Synthesis of Gelatin-Poly(ethylene glycol)-Tyramine (GPT)

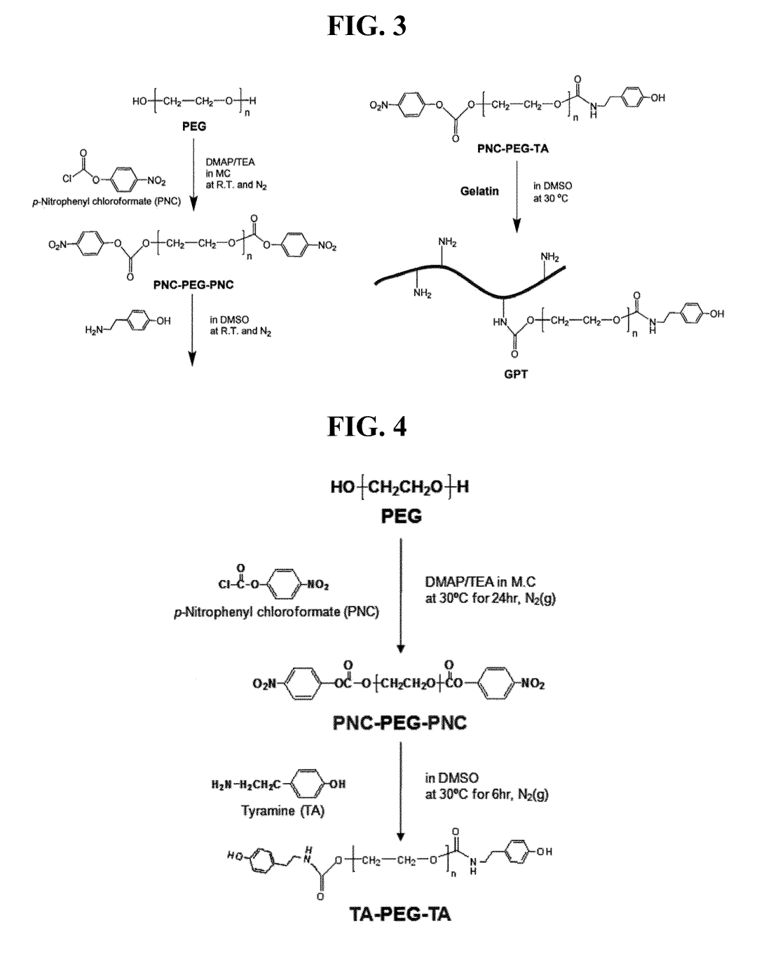

FIG. 3 is a reaction scheme showing the synthesis of GPT.

1. PEG-PNC Synthesis

To a solution of 10 g (2.9 mmol) of PEG in 100 mL of MC were added a solution of 0.779 g (6.38 mmol) of 4-dimethylaminopyridine (DMAP) and 0.645 g (6.38 mmol) of triethylamine (TEA) in 10 mL of MC and a solution of 1.286 g (6.38 mmol) of PNC in 50 mL of MC in the order, with the molar ratio of PEG:DMAP:TEA:PNC being 1:2.2:2.2:2.2. A reaction was performed at 30° C. for 24 hrs in a nitrogen atmosphere.

After completion of the reaction, the reaction mixture was filtered to remove the remaining reagents and then concentrated using a rotary evaporator. The concentrate was dropwise added to 1600 mL of chilled ether to form precipitates, which were then obtained by filtration. The filtrate was allowed to stand for 24 hrs in a vacuum oven to remove the remaining organic solvents to afford the desired compound (PEG-PNC) as a white powder.

2. Synthesis of gelatin-poly(ethylene glycol)-tyramine (GPT)

To a solution of 5 g (1.471 mmol) of PEG-PNC in 100 mL of DMSO was added a solution of 0.202 g (1.471 mmol) of TA in 50 mL of dimethylsulfoxide (DMSO), with the molar ratio of PEG-PNC:TA being 1:1. They were reacted at 30° C. for 6 hrs in a nitrogen atmosphere. Thereafter, a gelatin solution (1 g/200 ml in DMSO) was added and reacted at 30° C. for 24 hr in a nitrogen atmosphere.

After completion of the reaction, the reaction mixture was subjected to membrane dialysis against water (cutoff Mw 6000-8000 Da) to remove unreacted PEG-TA. The dialyzed solution was lyophilized to produce the desired compound (GPT) as a white powder. On a ¹HNMR spectrum, peaks appeared at 6.91-7.23 ppm which correspond to the TA substituents, confirming the synthesis of GPT.

### Synthesis Example 3

### Synthesis of Tyramine-Poly(ethylene glycol)-Tyramine(PEG-TA)

FIG. 4 is a reaction scheme showing the synthesis of a tyramine-poly(ethylene glycol)-tyramine(PEG-TA) copolymer.

1. Synthesis of PEG-PNC

To a solution of 10 g (2.9 mmol) of PEG in 100 mL of MC were added a solution of 0.779 g (6.38 mmol) of 4-dimethylaminopyridine (DMAP) and 0.645 g (6.38 mmol) of triethylamine (TEA) in 10 mL of MC and a solution of 1.286 g (6.38 mmol) of PNC in 50 mL of MC in the order, with the molar ratio of PEG:DMAP:TEA:PNC being 1:2.2:2.2:2.2. A reaction was performed at 30° C. for 24 hrs in a nitrogen atmosphere.

2. Synthesis of tyramine-poly(ethylene glycol)-tyramine (PEG-TA)

To a solution of 5 g (1.25 mmol) of PEG-PNC in 100 mL of methylene chloride (MC) was added 0.383 g (2.75 mmol) of tyramine (TA) in 100 mL of MC, with the molar ratio of PEG-PNC:TA being 1:2.2. A reaction was conducted at 30° C. for 6 hrs in a nitrogen atmosphere.

After completion of the reaction, the reaction mixture was filtered to remove remaining reagents and then concentrated using a rotary evaporator. The concentrate was dropwise added to 1600 mL of chilled ether to form precipitates, which were then obtained by filtration. The filtrate was allowed to stand for 24 hrs in a vacuum oven to remove the remaining organic solvents to afford the desired compound (PEG-TA) as a white powder.

### Synthesis Example 4

### Synthesis of methoxy poly(ethylene glycol)-tyramine (mPTA)

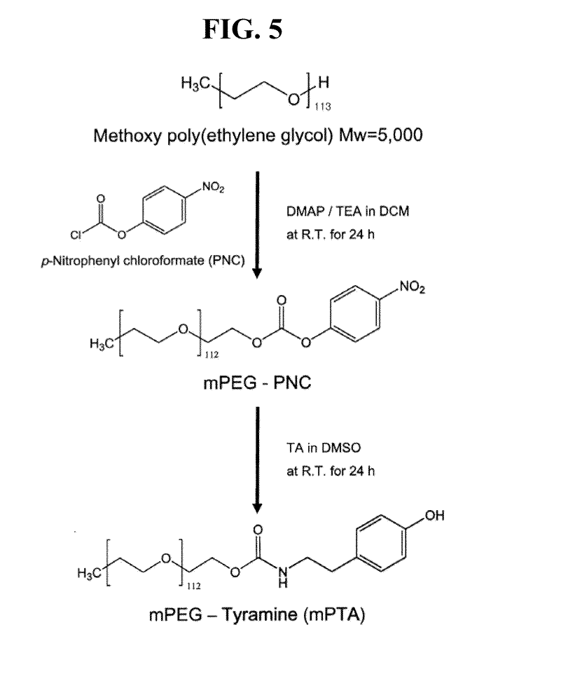

FIG. 5 is a reaction scheme showing the synthesis of methoxy poly(ethylene glycol)-tyramine (mPTA) copolymer.

1. Syntheis of mPEG-PNC

To a solution of 10 g (2 mmol) of PEG in 100 mL of MC were added a solution of 0.367 g (3 mmol) of 4-dimethylaminopyridine (DMAP) and 0.304 g (3 mmol) of triethylamine (TEA) in 20 mL of MC and a solution of 0.605 g (3 mmol) of PNC in 20 mL of MC in the order, with the molar ratio of PEG:DMAP:TEA:PNC being 1:1.5:1.5:1.5. A reaction was performed at RT for 24 hrs in a nitrogen atmosphere.

2. Synthesis of mPTA

To a solution of 7.8 g (1.5 mmol) of PEG-PNC in 100 mL of dimethyl sulfoxide (DMSO) was added 0.311 g (2.3 mmol) of tyramine (TA) in 50 mL of DMSO, with the molar ratio of PEG-PNC:TA being 1:1.5. A reaction was conducted at RT for 24 hrs in a nitrogen atmosphere.

After completion of the reaction, the reaction mixture was filtered to remove remaining reagents, after filtration, the reaction mixture was subjected to membrane dialysis against MeOH and acetone (cutoff Mw 3500 Da) to remove unreacted TA, and then the solution was concentrated using a rotary evaporator. The concentrate was dropwise added to 1600 mL of chilled ether to form precipitates, which were then obtained by filtration. The filtrate was allowed to stand for 24 hrs in a vacuum oven to remove the remaining organic solvents to afford the desired compound (mPTA) as a white powder.

### Example 1

### Conversion of Tyramine into Dopamine Using Tyrosinase

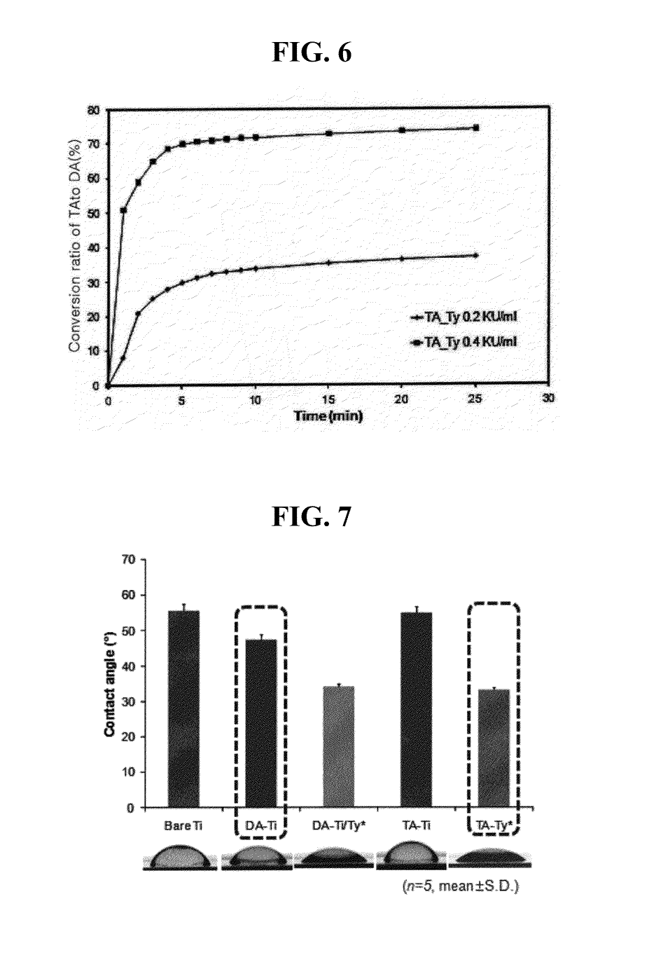

FIG. 6 is of saturation curves for tyramine showing the relationship between time and conversion ratio.

1 mL of tyramine having a concentration of 10 mg/mL was placed in a quartz cuvette and treated with tyrosinase (0.2 and 0.4 KU/mL). One minute after the enzymatic treatment, the conversion of tyramine dopamine was monitored for 30 min using UV/VIS sprectometer (JASXO, V-750 UV/VIS/NIR, Japan) (wavelength 270 nm).

As seen in the curves, the conversion rate from tyramine to dopamine could be adjusted according to the concentration of tyrosinase. Tyrosinase reached a conversion ratio of about 70% within 5 min when tyrosinase was used at a concentration of 0.4 KU/mL.

Taken together, the data show that tyrosinase can convert tyramine into dopamine/dopaquinone. Also, it was found that the dopamine/dopaquinone molecules converted from tyramine could be immobilized onto polymer or metal surfaces via coordinate bonds.

### Example 2

### Comparison Between Immobilization Techniques Using Dopa and Tyrosinase

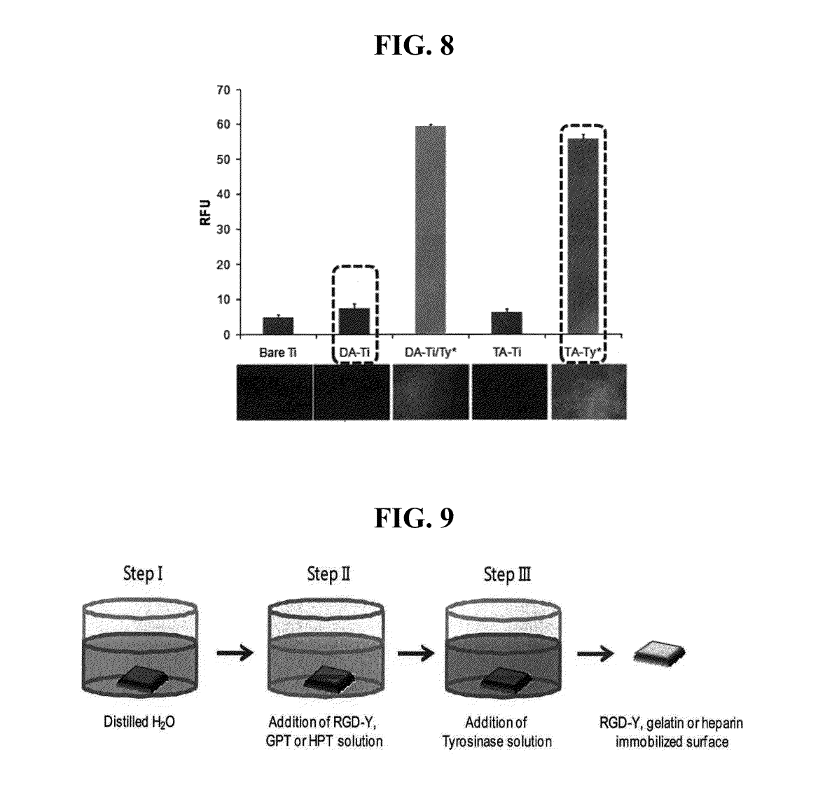

FIG. 7 is a graph showing contact angles on various metal specimens including a dopamine-immobilized metal specimen and a metal specimen on which tyramine was immobilized in the presence of tyrosinase. In FIG. 8, amine distributions over the metal specimens are depicted.

As illustrated in Example 1, tyramine molecules could be effectively converted into dopa/dopaquinone molecules. A comparison was made between conventional surface immobilization techniques using dopa and tyrosinase.

For experiments, dopamine and tyramine/tyrosinase were immobilized onto metal surfaces. In this regard, 450 μL of phosphate saline buffer was added to a piece of stainless steel or titanium metal and mixed with 40 μL of dopamine or tyramine having a concentration of 100 μg/mL. In the case of tyramine, tyrosinase having a concentration of 0.4 KU/mL was added in an amount of 10 μL. For effective comparison, a reaction was performed for 30 min. After immobilization, the specimens were washed three to five times with distilled water. An analysis was made of the results using contact angles and amine distributions on the surface. For contact angle measurements, droplets of 1 μL were deposited on a specimen surface and the angle at which the droplet interface meets the surface was measured using a contact angle meter (GBX Inc., France) and image analysis software.

The surface immobilized with tyramine in the presence of tyrosinase was found to become more hydrophilic than did the surface immobilized with dopamine, as the contact angle was measured to be 30° for tyramine and 45° for dopa, demonstrating that more effective surface immobilization can be achieved with tyramine in the presence of tyrosinase than with dopa.

To examine the surface distribution of amine resulting from the immobilization, FITC was reacted with amine on the surface, followed by observation under a fluorescence microscope. In this regard, dopamine- or tyramine-immobilized metal was treated for 1 hr with 1 mL of an FITC solution (100 μg/mL in ethanol) to induce FITC to couple with the amine immobilized onto the surface. After completion of the coupling reaction, the metal was washed three to five times with distilled water and ethanol to remove the FITC molecules that remained unreacted.

Fluorescence microscopic observation detected a fluorescent intensity of 55 RUF on the specimen immobilized using tyrosinase and showed an even distribution of green fluorescence over the specimen.

Therefore, the immobilization using tyrosinate was proven as being more effective than that using dopa.

### Example 3

### Immobilization of RGD-Y, GPT and HPT on Metal Surface Using Tyrosinase

FIG. 9 is a schematic illustration showing the immobilization of RGD, gelatin or heparin on a metal surface by simple immersion.

As a substrate for use in the immobilization of a bioactive molecule thereonto, a stainless steel or titanium piece was used. First, it was washed sequentially with propanol, ethanol and acetone. The stainless steel or titanium specimen was immersed in 450 μL of phosphate buffer saline to which 40 μL of a bioactive molecule solution was added at the following concentrations: 1) RGD-Y: 50-400 μg/mL, 2) HPT: 100-600 μg/mL, 3) GPT: 100-600 μg/mL. Thereafter, 10 μL of tyrosinase having a concentration of from 0.1 to 1 KU/mL was added to conduct an immobilization reaction at 37° C. for 5 min to 3 hrs with stirring at 100 rpm. A total volume of the reaction mixture was 500 μL.

The concentration of the immobilized bioactive molecule was adjusted depending on reaction time, the concentration of tyrosinase and the initial concentration of the bioactive molecule. After completion of the immobilization, the metal specimen was washed three to five times with distilled water and dried in a vacuum oven. The specimen was found to have the bioactive molecule immobilized thereonto.

### Example 4

### Immobilization of Growth Factor on Metal Surface Using Tyrosinase

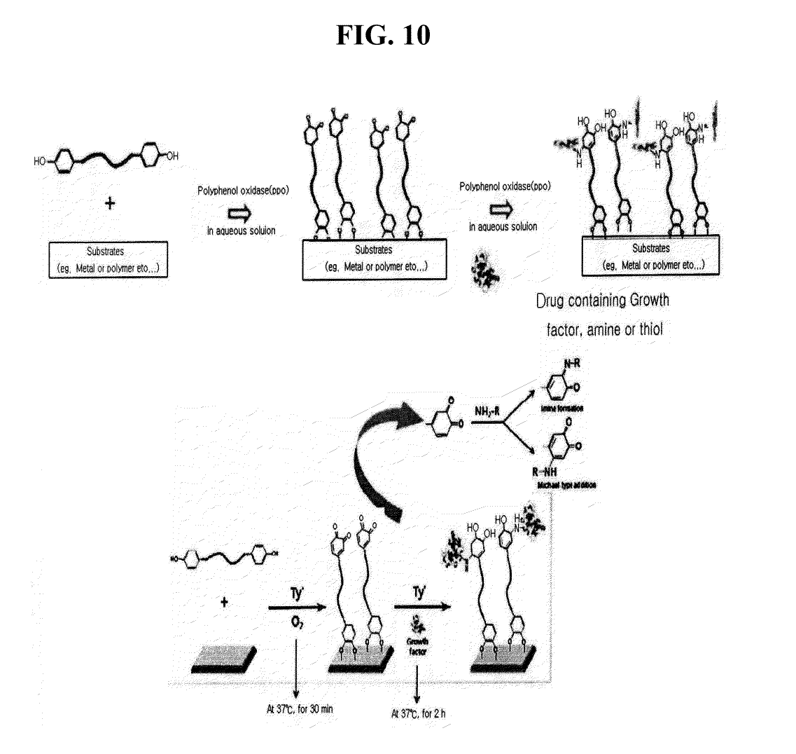

FIG. 10 is a schematic illustration showing the introduction of a protein drug (e.g. growth factor) onto a metal or polymer substrate by coupling with dopaquinone molecules which were formed when a tyramine-poly(ethylene glycol)-tyramine (PEG-TA) was anchored to the surface of the metal or polymer substrate in the presence of tyrosinase by a simple immersion procedure.

Tyramine-poly(ethylene glycol)-tyramine (PEG-TA) was anchored to a substrate using tyrosinase, with the concomitant formation of dopaquinone molecules. Then, the dopaquinone molecules were coupled with a growth factor so that it was immobilized on the substrate.

PEG-TA was prepared from PEG with a molecular weight of 4,000 Da as described in Synthesis Example 3. A stainless steel or titanium specimen was placed in 490 μL of PEG-TA to which 10 μL of tyrosinase was then added at a concentration of 0.4 KU/mL, followed by immobilization reaction for 30 min. The PEG-TA-anchored specimen was washed three to five times with distilled water before 500 μL of a growth factor or a protein drug was added at a concentration of from 1 to 50 μg/mL to induce coupling between the amine or thiol groups of the growth factor or protein drug and the dopaquinone molecules formed at the terminus of the PEG moiety. PEG-TA was used at a concentration of from 10 to 100 μg/mL.

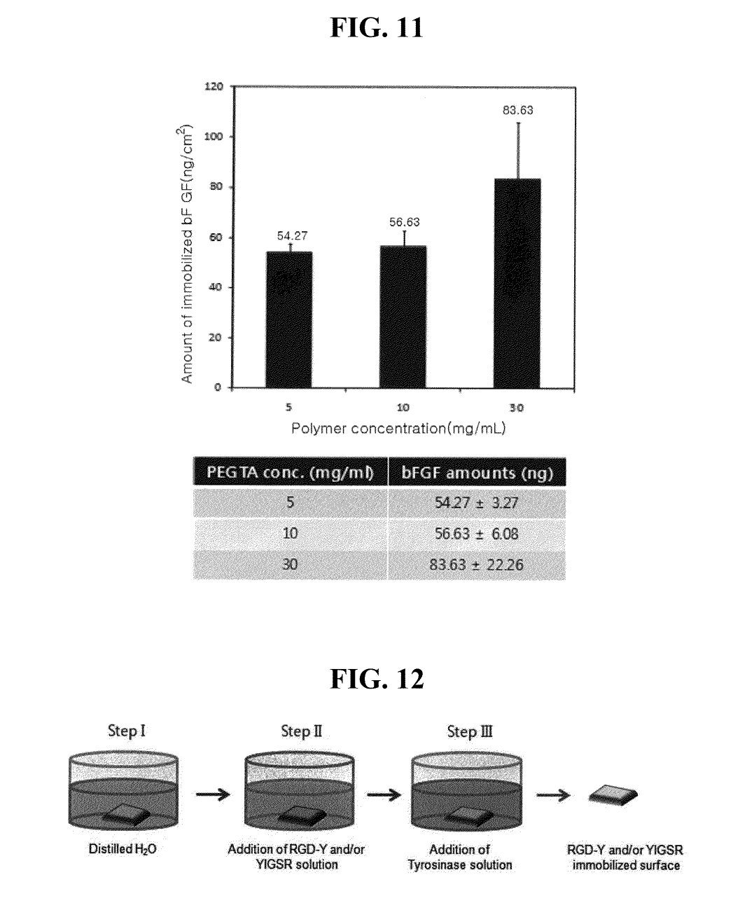

As illustrated in FIG. 11, the growth factor was found to be effectively introduced onto the substrate surface through the mediation of tyrosinase action. The concentration of the immobilized growth factor was adjusted depending on the concentrations of PET-TA, tyrosinase and the initially used growth factor.

### Example 5

### Immobilization of RGD-Y and YIGSR on Polymer Surface Using Tyrosinase

FIG. 12 is a schematic illustration showing the immobilization of the peptides RGD and YIGSR on a polymer (polyurethane) surface by a simple immersion procedure using tyrosinase.

For use as a substrate in immobilizing the peptides RGD and YIGSR, which facilitates endothelialization, thereonto using tyrosinase, a polymeric mesh (polyurethane) was used. The polymeric mesh was punched to form a hole 1 cm in diameter and immersed in 450 μL of phosphate buffer saline to which 40 μL of an RGD or YIGSR solution was then added. The peptide was used in an amount of 50-400 μg. Thereafter, 10 μL of tyrosinase having a concentration of 0.4 KU/mL was added to conduct an immobilization reaction at 37° C. for 5 min to 3 hrs. A total volume of the reaction mixture was set to be 500 μL. The polyurethane was found to have the peptide immobilized onto the surface thereof.

### Example 6

### Immobilization of mPTA on the Versatile Surfaces Using Tyrosinase

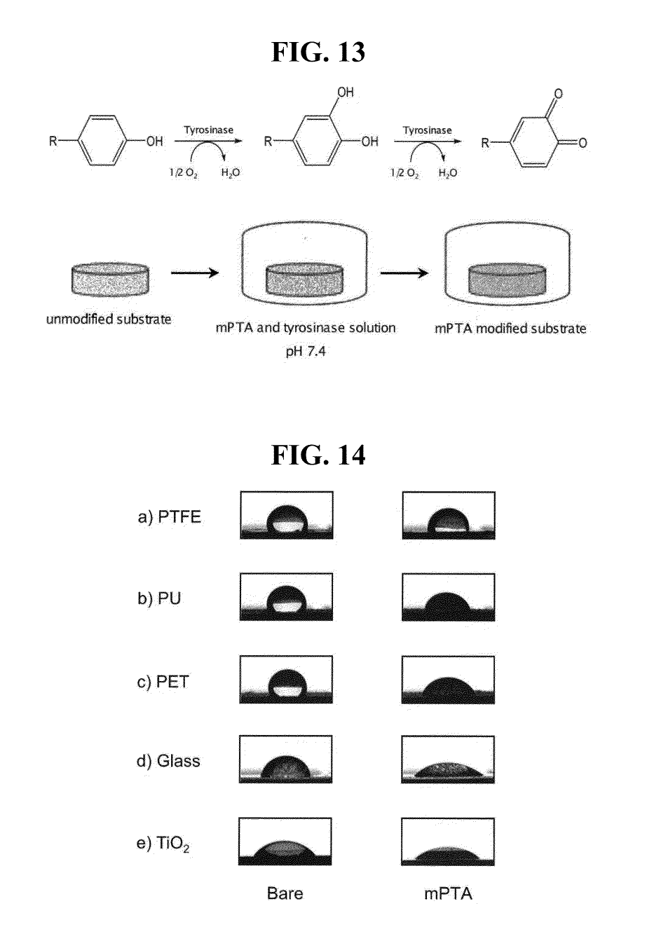

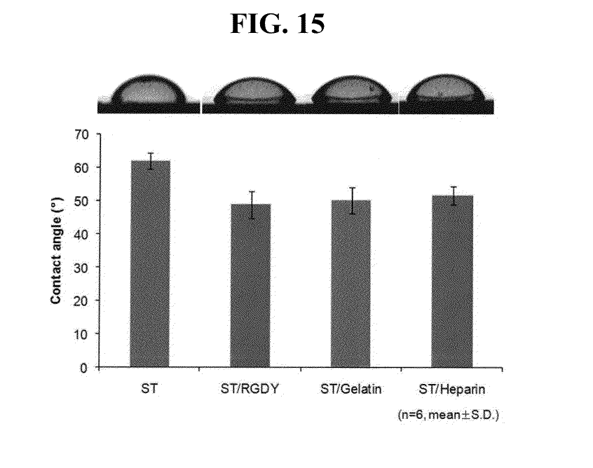

FIG. 13 is a schematic illustration showing the immobilization of mPTA on versatile surfaces by simple immersion using tyrosinase.

As a substrate for use in the immobilization of a mPTA, a polyethylene terephthalate (PET), polyurethane (PU), Teflon (PTFE), glass or titanium piece was used. First, it was washed sequentially by sonication. The versatile specimens were immersed in 450 μL of phosphate buffer saline to which 40 μL of mPTA (10˜100 mg/mL) was added. Thereafter, 10 μL of tyrosinase having a concentration of from 0.1 to 1 KU/mL was added to conduct an immobilization reaction at 37° C. for 5 min to 3 hrs with stirring at 100 rpm. A total volume of the reaction mixture was 500 μL.

The concentration of the immobilized mPTA was adjusted depending on reaction time, the concentration of tyrosinase and the initial concentration of the bioactive molecule. After completion of the immobilization, the specimens were washed three to five times with distilled water and dried in a vacuum oven. The specimen was found to have the bioactive molecule immobilized thereonto.

### Example 7

### Measurement of Contact Angle on Substrate Surface Immobilized with Bioactive Molecule

Contact angles were measured to examine the change in hydrophilicity of bioactive molecule-immobilized surface. Droplets of 1 μL were deposited on a specimen surface after which contact angles were measured using a contact anglemeter (GBX Inc., France) equipped with image analysis software.

The results are graphed in FIG. 14, 15. As seen in FIG. 14, 15, the water contact angle was measured to be about 62° and about 49˜51° on the specimens immobilized without and with the bioactive molecule, respectively, indicating that relatively hydrophilic RGD or heparine molecules were immobilized onto the surface.

### Example 8

### Quantitative Analysis of Bioactive Molecule Immobilized onto Substrate

To examine the effect of concentrations of tyrosinase and initial RGD and reaction time on RGD immobilization, experiments were conducted with RGD according to various conditions. RGD immobilized on the surface was quantitatively analyzed using a fluorescamine assay. In this regard, a RGD-immobilized specimen was placed in 375 μL of phosphate buffer saline to which 125 μL of a fluorescamine solution (100 μg/mL in acetone) was then added. After reaction for 1 min, the reaction mixture was transferred to 96 well-plates to measure fluorescent intensity with excitation at 390 nm and emission at 475 nm.

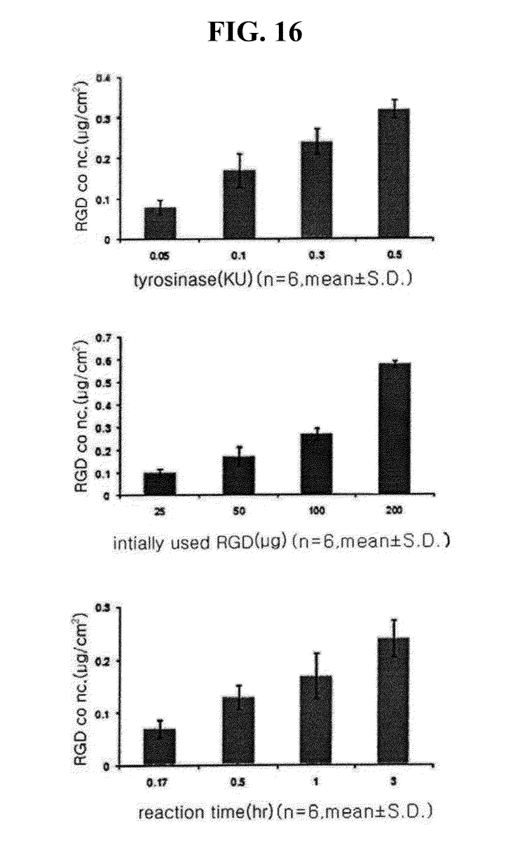

As seen in FIG. 16 where the results are depicted, the concentration of the immobilized RGD was found to increase with escalation of initial RGD amount and reaction time. The concentration of the immobilized RGD could be adjusted to a concentration of from about 0.08 to 0.58 μg/cm². In practice, only when immobilized at a concentration of 0.17 μg/cm² or higher, RGD was found to exhibit effective cell adhesion activity as analyzed by a cell assay.

In addition, an examination was made of effects of the initial amount of tyrosinase, HPT and reaction time on the immobilization of heparin. To this end, experiments for HPT surface immobilization were set on various conditions, followed by a toluidine blue assay for the quantitative analysis of heparin on the surface. A specimen immobilized with heparin was treated at room temperature for 30 min with 500 μL of toluidine blue dye (0.005% solution), during which stirring facilitated the formation of a heparin-toluidine complex. After the mixture was shaken with 3 mL of hexane to remove the heparin-toluidine complex by adsorption at the interface, the aqueous phase in which toluidine blue remained uncombined was analyzed by UV at 630 nm. Heparin was used at a concentration of from 0.1 to 8 μg/mL to draw a calibration curve.

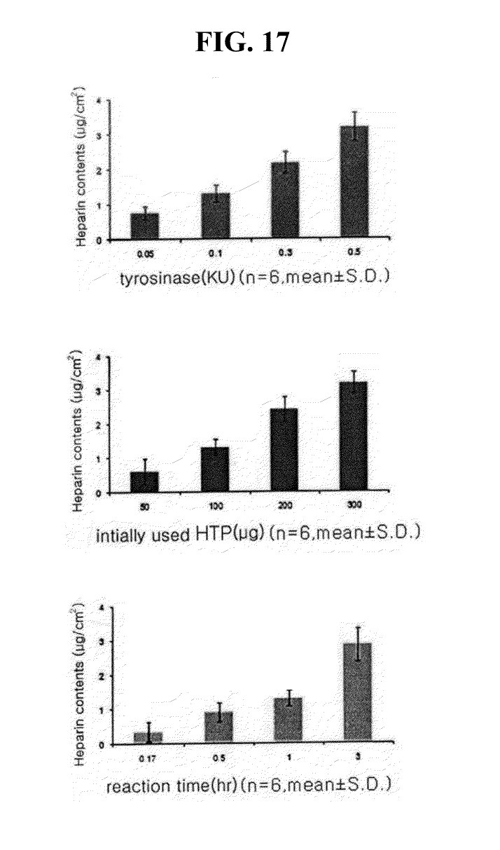

As seen in FIG. 17 where the results are depicted, the concentration of the immobilized heparin was found to increase with increasing of tyrosinase, initial HPT amount and reaction time. The concentration of the immobilized heparin could be adjusted to a concentration of from about 0.35 to 3.21 μg/cm².

To examine the effect of concentrations of tyrosinase and initial GTP, and reaction time on gelatin immobilization, surface immobilization experiments were conducted with GPT according to various conditions. Gelatin immobilized on the surface was quantitatively analyzed using a BCA kit. In this regard, a gelatin-immobilized specimen was treated at room temperature for 4 hrs with 500 μL of a BCA, after which the reaction mixture was transferred to 96-well plates and measured for fluorescent intensity.

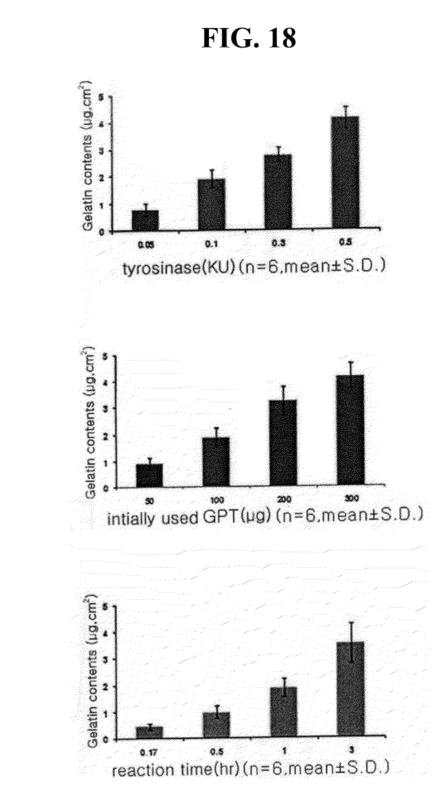

As seen in FIG. 18 where the results are depicted, the concentration of the immobilized gelatin was found to increase with escalation of initial GPT amount and reaction time. The concentration of the immobilized gelatin could be adjusted to a concentration of from about 0.45 to 3.81 μg/cm².

In order to examine the immobilization of the peptides RGD and YIGSR in the presence of tyrosinase, they were immobilized on polyurethane meshes, followed by a fluorescamine assay for quantitatively analyzing the RGD on the surface. In this context, RGD-immobilized specimen was placed in 375 μL of phosphate buffer saline to which 125 μL of a fluorescamine solution (100 μg/mL in acetone) was then added. After reaction at room temperature for 1 min, the reaction mixture was transferred to 96 well-plates to measure fluorescent intensity with excitation at 390 nm and emission at 475 nm.

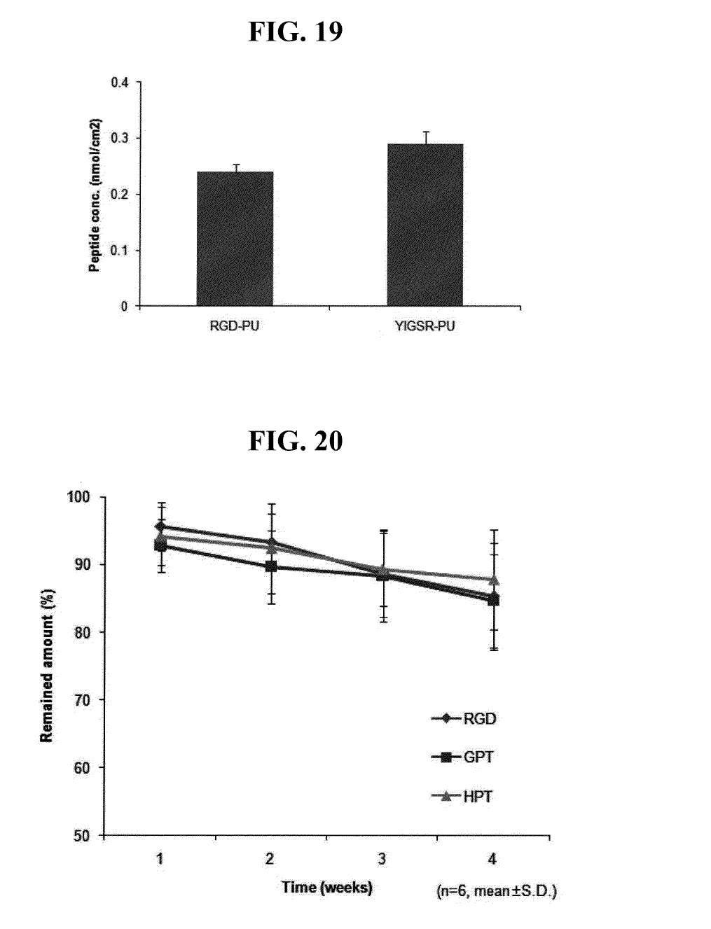

As seen in FIG. 19 where the results are depicted, each of RGD and YIGSR was found to be immobilized at a concentration of 0.2 nmol/cm² orhigheronthepolyurethanemeshes.

### Example 9

### Assay of Immobilized Bioactive Molecule for Surface Stability

The stainless steel on which the bioactive molecules of Example 3 were immobilized was incubated at 37° C. for 30 days in 0.01 M phosphate buffer saline in an incubator. On day 0 to day 30, their in vitro stability was assayed by quantitatively analyzing RGD, gelatin and heparin retained on the surfaces using a fluorescamine assay, a BCA kit, and a toluidine blue assay, respectively.

Even after storage for one month, as seen in FIG. 20 where the results are depicted, the RGD, gelatin and heparin retained as much as 70˜80% of their initial amounts.

### Example 10

### In Vitro Assay of Immobilized Bioactive Molecule for Cell Attachment and Proliferation

For in vitro cell attachment assay, osteoblasts (MC3T3-E1) were cultured at a density of 2×10⁴ cell/cm² for 2 hrs or overnight on RGD- or gelatin-immobilized stainless steel, titanium or polyurethane plates, followed by conducting an MTTassay. The MTT assay is a colorimetric assay for representing cell viability as optical density, thus allowing assessment of the adhesion or proliferation of cells. Also, an F-actin assay was performed to visualize the morphology of cultured cells. The cells cultured on the surface were fixed with 1 mL of paraformaldehyde (4%), and treated with rhodamine and DAPI for cytoplasmic and nuclear staining, respectively. The stained cells were visualized using a fluorescent microscope.

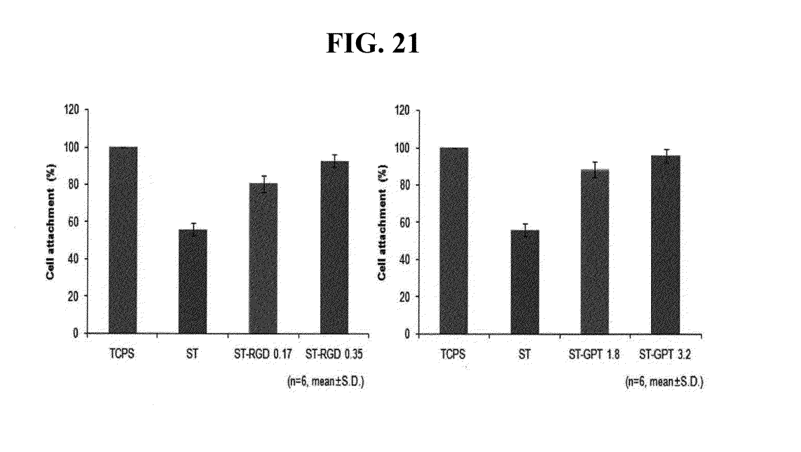

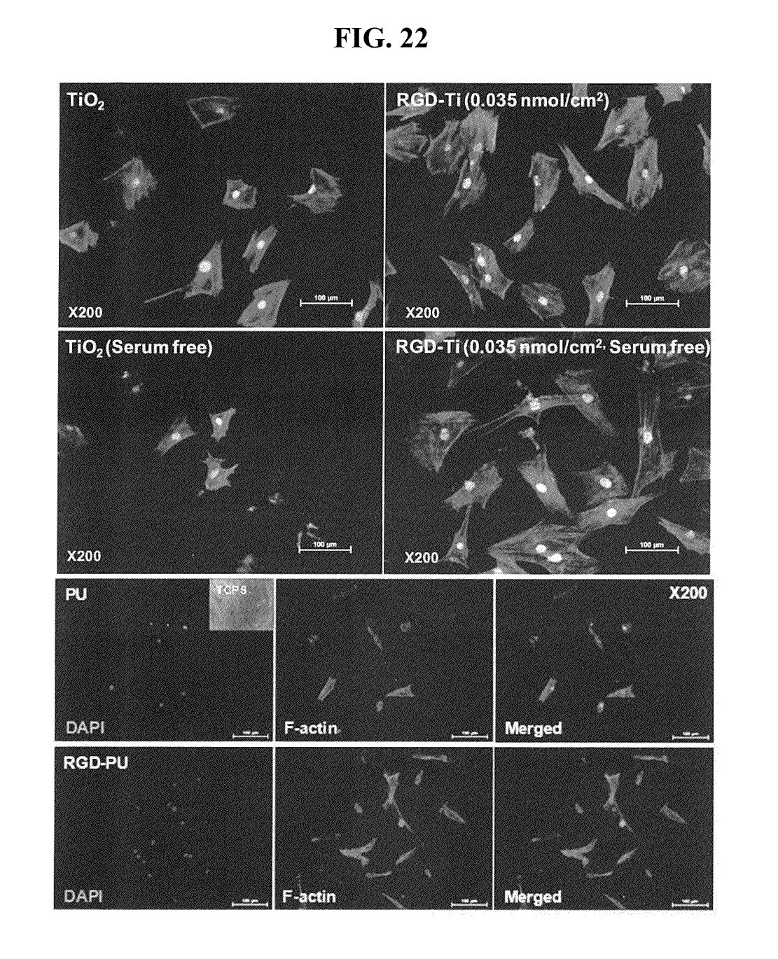

As seen in FIG. 21, RGD- or gelatin-immobilized stainless steel, titanium or polyurethane was increased in cell attachment capacity. The osteoblasts were found to be attached to the RGD- or gelatin-immobilized metal or polymer at a rate of as high as about 80˜90% while the bare metal or polymer allowed a cell attachment rate of about 50˜60% only. Morphologies of the cells cultured on the metal surface are given in FIG. 22. As seen in the flouresecence microphotographs, the cells were longer in cytoplasm length or larger in cytoplasm area when they were cultured on the surface to which bioactive molecules were immobilized using tyrosinase.

This was attributable to the fact that the RGD or gelatin which was stably introduced onto a metal surface using tyrosinase improves the attachment of osteoblasts. Thus, it was found that the use of tyrosinase allowed the stable immobilization of bioactive molecules on metal surfaces and that the immobilized bioactive molecules could retain their activity and help cells attach thereto.

For cell proliferation assay, osteoblasts (MC3T3-E1) were cultured at a density of 1×10⁴ cell/cm² for 7 days on RGD- or gelatin-immobilized stainless steel, titanium or polyurethane surfaces, followed by conducting an MTTassay. Each specimen is represented in the form of, for example, ST-RGD 0.17 which stands for the immobilization of RGD at a concentration of 0.17 μg/cm² on stainless steel.

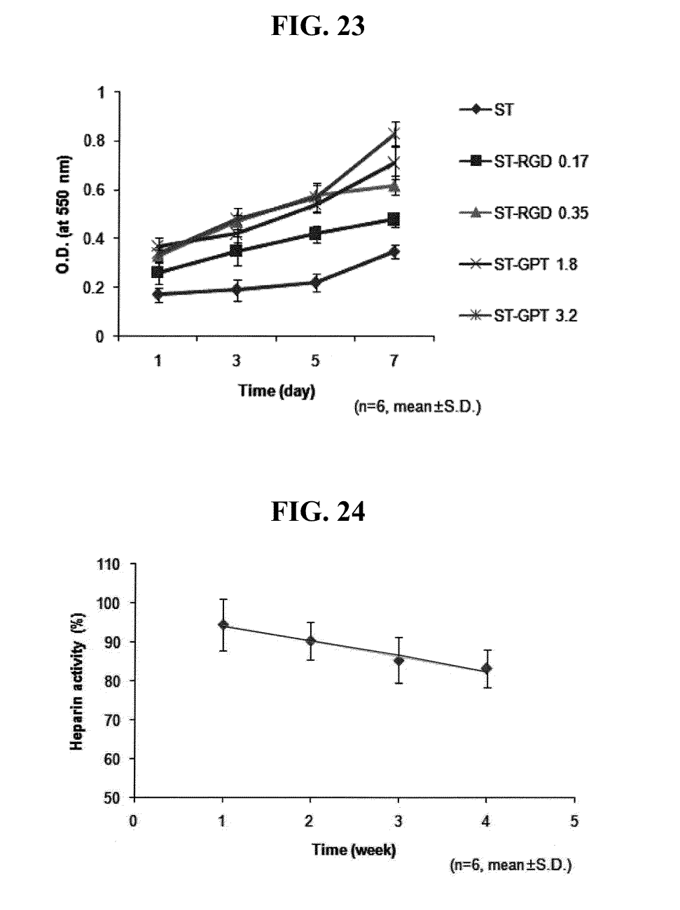

As seen in FIG. 23, the RGD- or gelatin-immobilized stainless steel improved the proliferation of osteoblasts (MC3T3-E1). With the improvement in initial cell attachment, the surface on which RGD or gelatin was immobilized using tyrosinase guaranteed higher cell proliferation than did the bare surface.

Hence, the simple technique of using tyrosinase to immobilize cell adhesion peptides or proteins could be useful for increasing cell activity on biomaterial surfaces.

### Example 11

### Assay of Immobilized Heparin for Activity

To examine the activity of the heparin which was immobilized on a surface with the aid of tyrosinase, a Factor Xa assay was conducted. To this end, heparin-immobilized metal surfaces were incubated at 37° C. for 4 weeks in 0.01 M phosphate buffer saline in an incubator. During the incubation for 4 weeks, the heparin remaining on the surface was examined for activity using a Factor Xa assay, with free heparin serving as a control.

The results are given in FIG. 24. As seen in the plot of FIG. 24, the immobilized heparin was found to retain 80˜85% of the initial activity. Therefore, the simple technique of using tyrosinase in immobilizing a bioactive molecule, such as heparin, on a surface in accordance with the present invention may be useful for coating medical devices applicable to the vascular system.

### Example 12

### In Vivo Assay of Artificial Blood Vessels Made of Polyurethane on which Heparin, RGD or YISGR was Immobilized with the Aid of Tyrosinase

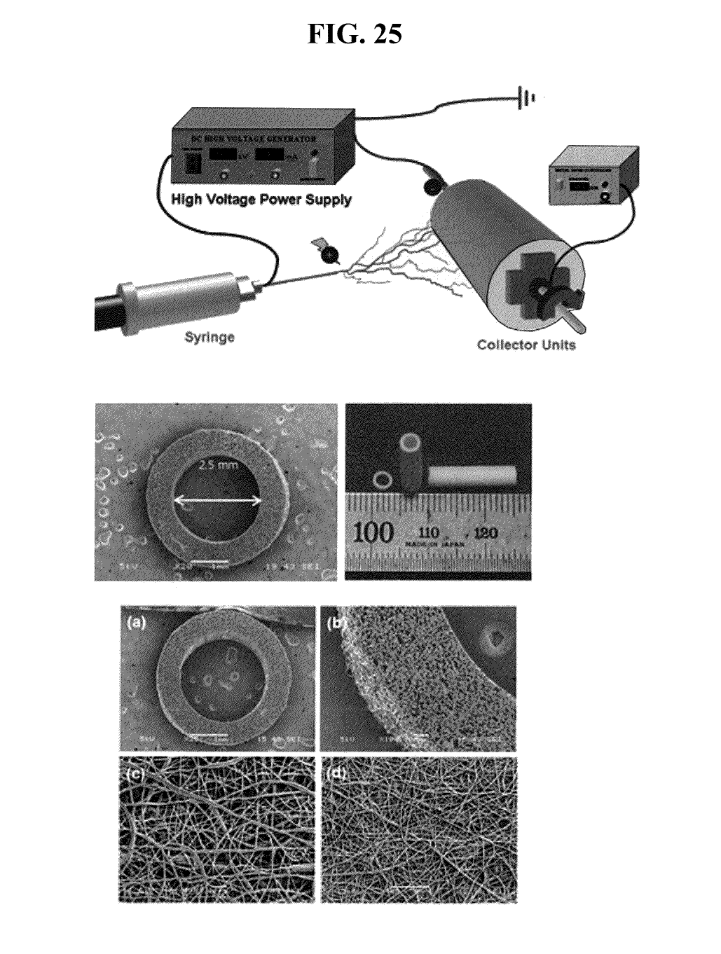

FIG. 25 is a schematic illustration showing the preparation of artificial blood vessels made of polyurethane by electrospinning, together with electron microphotographs showing structures of the artificial blood vessels.

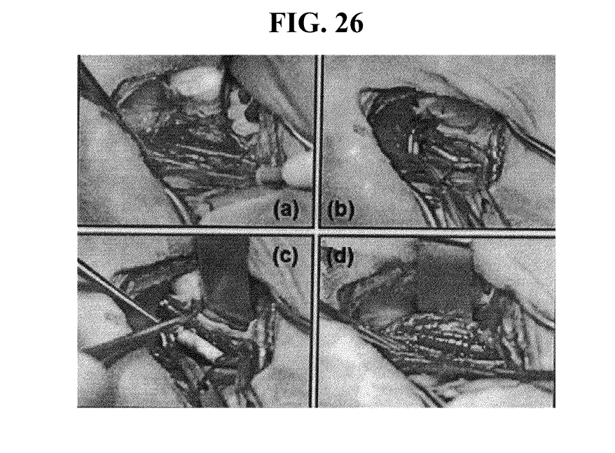

FIG. 26 is of photographs showing an animal experiment using a bioactive molecule (heparin, RGD or YIGSR)-immobilized artificial blood vessel made of polyurethane.

For use in the experiment, artificial blood vessels were prepared from polyurethane by electrospinning. Conditions for this electrospinning included a polymer concentration of 20%, a spinning speed of 10 μL, a spinning time of 3 hrs, a voltage of 10 kV, a collector speed of 400 rpm, and a collector-to-tip distance of 25 cm.

Rabbits were employed as animal models. They were given a general anesthetic and endotracheal intubation and allowed to breathe with the aid of a mechanical respiratory system. The whole abdomen was shaved and sterilized twice with Betadine. The abdomen was longitudinally opened at the mid line to expose the carotid artery, and heparin was intravenously injected at a dose of 1˜3 mg. After the ligature of the carotid artery at distal and proximal regions with clamps, the cartotid artery was cut at a length of 2 cm. A polyurethane artificial blood vessel on which a bioactive molecule and a cell adhesion peptide were immobilized was connected to the resected carotid artery by end-to-end anastomosis with Prolene 6-0 suture first at the proximal site and then at the distal site. Following deaeration, the clamps were removed one by one. When no hemorrhages were detected at the anastomotic sites, the wound was sutured. After the rabbits returned back to spontaneous respiration, the intubation was removed and they were transferred to breeding cages. The rabbits were administered with antibiotics and analgesics just before and after the operation, and twice a day.

For excision assay, Doppler sonography was first performed once a week to examine the bloodcommunication through the artificial blood vessel implant. For a predetermined period after the operation, the artificial blood vessels were photographed to monitor the bloodflow therethrough and the morphology thereof. When the occlusion of the artificial blood vessel or paraplegia was observed, the rabbits were euthanized. The rabbits which maintained blood communication through the artificial blood vessel for four months after the operation were also euthanized. After being excised from the euthanized rabbits, the artificial blood vessel implants were observed for histological properties by H&E stain and various immune-stains against vWF, SMA, CD68 and CD31. In this context, the excised artificial blood vessels were partially cut and immunostained against CD31 and vWF to examine blood endothelial cells, against the α-chain and myosin heavy chain of smooth muscle cells to examine smooth muscles, and against CD68 to examine macrophages. Electron microscopy was also used to observe the formation of endothelial cells and smooth muscle cells in the excised blood vessels. Blood communication according to regions was also examined with an image analyzer.

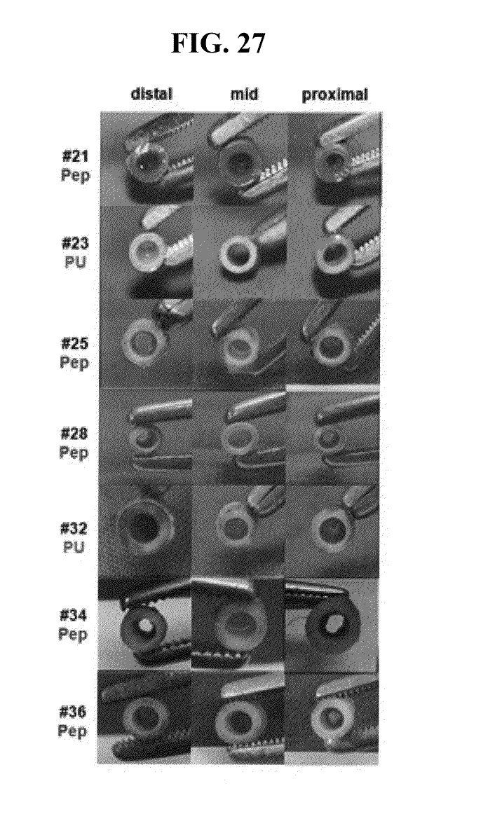

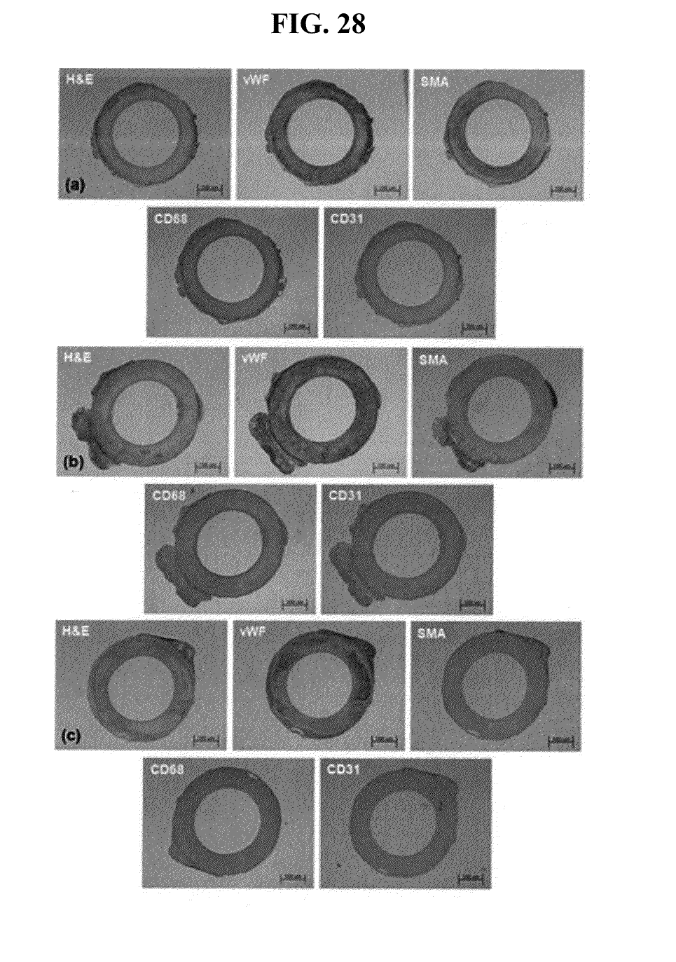

As seen in FIG. 27, no significant stenosis was incurred in the artificial blood vessel implants. The regions at which stenosis occurred witin the vessel were immunostained against endothelial cells (Factor 8, CD31), smooth muscle cells (smooth muscle alphaactin), and macrophage (CD68). As a result, as shown in FIG. 28, endothelial cells were observed PU vessels immobilized with peptides or CD34 antibody and heparin, compared to bare PU vessels.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### INDUSTRIAL APPLICABILITY

This invention is provided for immobilizing a bioactive molecule onto a surface using polyphenol oxidase. Based on the surface immobilization of bioactive molecules using polyphenol oxidase, various bioactive molecules such as osteogenetic peptides and growth factors can be simply immobilized to medical metal or polymer substrate surfaces such as orthopedic or dental implants which can be then effectively used to induce rapid osteogenesis after being transplanted. Also, antithrombotic agents and/or entothelialization inducing agents may be immobilized to medical substrates for vascular systems, such as stents and artificial blood vessels, thus guaranteeing hemo-compatibility to the medical substrates.

### SEQUENCE LISTING FREE TEXT

SEQ ID NO. 1 (RGD-Y) is a cell adhesion peptide.

SEQ ID NO. 2 (KQAGDV-Y) is a cell adhesion peptide.

SEQ ID NO. 3 (YIGSR) is a cell adhesion peptide.

SEQ ID NO. 4 (REDV-Y) is a cell adhesion peptide.

SEQ ID NO. 5 (IKVAN-Y) is a cell adhesion peptide.

SEQ ID NO. 6 (RNIAEIIKDI-Y) is a cell adhesion peptide.

SEQ ID NO. 7 (KHIFSDDSSE-Y) is a cell adhesion peptide.

SEQ ID NO. 8 (VPGIG-Y) is a cell adhesion peptide.

SEQ ID NO. 9 (FHRRIKA-Y) is a cell adhesion peptide.

SEQ ID NO. 10 (KRSR-Y) is a cell adhesion peptide.

SEQ ID NO. 11 (NSPVNSKIPKACCVPTELSAI-Y) is a cell adhesion peptide.

SEQ ID NO. 12 (APGL-Y) is a cell adhesion peptide.

SEQ ID NO. 13 (VRN-Y) is a cell adhesion peptide.

SEQ ID NO. 14 (AAAAAAAAA-Y) is a cell adhesion peptide.

SEQ ID NO. 15 (GRGDGGGGGY) is a cell adhesion peptide.

1. A method for immobilization of a bioactive molecule on a surface, comprising:
preparing a phenol-, catechol- or its derivative containing bioactive molecule (step 1); and treating a substrate surface with the bioactive molecule in presence of polyphenol oxidase to immobilize the bioactive molecule onto the surface (step 2).

2. The method according to claim 1, wherein the bioactive molecule contains a cell adhesion peptide containing a tyrosine.

3. The method according to claim 2, wherein the cell adhesion peptide is selected from the group consisting of peptides of SEQ ID NO. 1 (RGD-Y), SEQ ID NO. 2 (KQAGDV-Y), SEQ ID NO. 3 (YIGSR), SEQ ID NO. 4 (REDV-Y), SEQ ID NO. 5 (IKVAN-Y), SEQ ID NO. 6 (RNIAEIIKDI-Y), SEQ ID NO. 7 (KHIFSDDSSE-Y), SEQ ID NO. 8 (VPGIG-Y), SEQ ID NO. 9 (FHRRIKA-Y), SEQ ID NO. 10 (KRSR-Y), SEQ ID NO. 11 (NSPVNSKIPKACCVPTELSAI-Y), SEQ ID NO. 12 (APGL-Y), SEQ ID NO. 13 (VRN-Y) and SEQ ID NO. 14 (AAAAAAAAA-Y) and a combination thereof.

4. The method according to claim 1, wherein the bioactive molecule comprises one or more polymers, represented by the following Chemical Formula 1, in which a phenol or catechol derivative is grafted via a linker or directly to a polymer backbone: 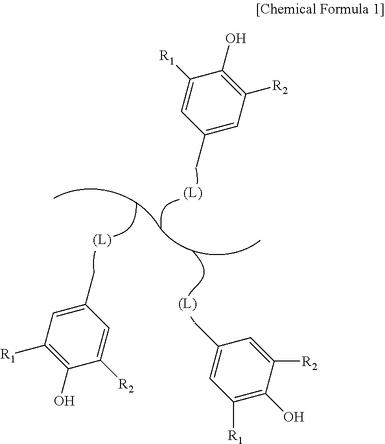
wherein, R₁ and R₂, which may the same or different, are independently hydroxyl or Hydrogen; and L is a polymeric linker.

5. The method according to claim 4, wherein the biomolecule is prepared by grafting a phenol or catechol derivative represented by the following Chemical Formula 2 to the polymer backbone having amino, hydroxyl or carboxyl groups through an amide, urethane, urea or ester bond, with a water-soluble polymer serving as the linker: 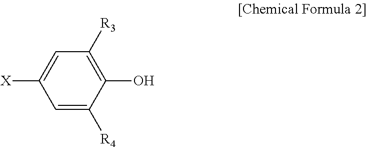
wherein, R₃ and R₄, which may the same or different, are independently hydroxyl or hydrogen, and X is a carboxyl group or anamine group.

6. The method according to claim 4, wherein the polymer backbone is derived from a polymer or one more selected from the group consisting of heparin, hyaluronic acid, collagen, gelatin, chitosan, cellulose, dextran, dextran sulfate, chondroitin sulfate, keratan sulfate, dermatan sulfate, alginate, albumin, fibronectin, laminin, elastin, vitronectin, fibrinogen, polyethylene glycol[PEG], polyethylene oxide[PEO], polyethylene imine[PEI], polypropylene oxide [PPO], polyvinyl alcohol [PVA], poly(N-isopropyl acrylamide) [polyNIPAM], polyfumarate, polyorganophosphagene, polyacrylic acid [polyAAc], polyacryl sulfonate, polyhydroxyethylmethacrylate [PolyHEMA], PEO-PPO-PEO (Pluronic® series), 4-arm PEO-PPO-PEO (Tetronic® series), PEG-PEI, PEG-PVA, PEG-PEI-PVA, PEI-PVA, poly(NIPAAM-co-AAc), poly(NIPAAM-co-HEMA), and combinations thereof.

7. The method according to claim 4, wherein the polymer backbone may be derived from a polymer or one more selected from the group consisting of fibroblast growth factor (FGF), vascular endothelial growth factor (VEGF), transforming growth factor (TGF), bone morphogenetic protein (BMP), human growth hormone (hGH), pig growth hormone (pGH), granulocyte colony-stimulating factor (G-CSF), erythropoietin (EPO), macrophage colony-stimulating factor (M-CSF), tumor necrosis factor (TNF), epidermal growth factor (EGF), platelet-derived growth factor (PDGF), interferon-α,β,γ, interleukin-2 (IL-2), calcitonin, nerve growth factor (NGF), growth hormone releasing hormone, angiotensin, luteinizing hormone releasing hormone (LHRH), luteinizing hormone releasing hormone agonist (LHRH agonist), insulin, thyrotropin-releasing hormone (TRH), angiostatin, endostatin, somatostatin, glucagon, endorphine, bacitracin, mergain, colistin, monoclonal antibodies, and vaccines.

8. The method according to claim 4, wherein the polymer backbone is derived from the group consisting of an anti-proliferative agent, an anti-inflammatory agent, an anti-thromobotic agent and a combination thereof, said anti-proliferative agent being selected from among sirolimus (rapamycin), everolimus, pimecrolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenphosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, smc proliferation inhibitor-2w, epothilone A and B, mitoxantrone, azathioprine, mycophenolatmofetil, c-myc-antisense, b-myc-antisense, betulinic acid, camptothecin, PI-88 (sulfated oligosaccharide), melanocyte stimulating hormone(α-MSH), activated protein C, IL-1β inhibitors, thymosine α-1, fumaric acid and its esters, calcipotriol, tacalcitol, lapachol, β-lapachone, podophyllotoxin, betulin, podophyllic acid 2-ethylhydrazide, molgramostim (rhuGM-CSF), peginterferon α-2b, lenograstimn (r-HuG-CSF), filgrastim, macrogol, dacarbazine, basiliximab, daclizumab, selectin (cytokine antagonist), CETP inhibitors, cadherines, cytokinin inhibitors, COX-2 inhibitors, NFkB, angiopeptin, ciprofloxacin, fluoroblastin, monoclonal antibodies inhibitive of mycocyte proliferation, bFGF antagonists, probucol, prostaglandin, 1,11-dimethoxycanthin-6-one, 1-hydroxy-11-methoxycanthin-6-one, scopoletin, colchicine, NO donors including pentaerythritol tetranitrate or syndnoeimine, S-nitroso derivatives, tamoxifen, staurosporine, β-estradiol, α-estradiol, estriol, estrone, ethinylestradiol, fosfestrol, medroxyprogesterone, estradiol cypionate, estradiol benzoate, tranilast, cancer therapeutic kamebakaurin and other terpenoids, verapamil, tyrosine kinase inhibitors (tyrphostines), cyclosporine A, paclitaxel and its derivatives such as 6-α-hydroxy-paclitaxel, baccatin, taxotere, macrocyclic oligomers (MCS) of natural or synthetic carbon suboxide and their derivatives, mofebutazone, acemetacin, diclofenac, lonazolac, dapsone, O-carbamoylphenoxyacetic acid, lidocaine, ketoprofen, mefenamicacid, piroxicam, meloxicam, chloroquine phosphate, penicillamine, tumstatin, avastin, D-24851, SC-58125, hydroxychloroquine, auranofin, sodium aurothiomalate, oxaceprol, celecoxib, β-sitosterin, ademetionine, myrtecaine, polidocanol, nonivamide, levomenthol, benzocaine, aescin, ellipticine, D-24851 Calbiochem, colcemid, cytochalasin A-E, indanocine, nocodazole, S 100 protein, bacitracin, vitronectin receptor antagonist, azelastine, guanidyl cyclase stimulator, tissue inhibitor of metal proteinase-1 and -2, free nucleic acids, nucleic acids, DNA and RNA fragments integrated into viral vectors, plasminogen activator inhibitor-1, plasminogen activator inhibitor-2, antisense oligonucleotides, VEGF inhibitors and IGF-1; said anti-inflammatory agent being selected from among natural or synthetic steroids including cefadroxil, cefazolin, cefaclor, cefotaxim, tobramycin, gentamycin, dicloxacillin, oxacillin, leflunomide, anakinra, etanercept, sulfasalazine, etoposide, dicloxacillin, tetracycline, triamcinolone, mutamycin, procainamid, D24851, SC-58125, retinoic acid, quinidine, disopyramide, flecamide, propafenone, sotalol, amidorone, bryophyllin A, inotodiol, maquiroside A, ghalakinoside, mansonine, strebloside, hydrocortisone, betamethasone and dexamethasone, nonsteroidal substances (NSAIDS) including fenoprofen, ibuprofen, indomethacin, naproxen and phenylbutazone, antiprozoal agents such as acyclovir, ganciclovir, zidovudine, clotrimazole, flucytosine, griseofulvin, ketoconazole, miconazole, nystatin, terbinafine, chloroquine, mefloquine and quinine, hippocaesculin, barringtogenol-C21-angelate, 14-dehydroagrostistachin, agroskerin, agrostistachin, 17-hydroxyagrostistachin, ovatodiolid, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-β-hydroxypregnadiene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-AII, bisparthenolidine, periplocoside A, ghalakinoside, ursolic acid, deoxypsorospermin, sychorubin, ricin A, sanguinarine, manwu wheat acid, methylsorbifolin, sphatheliachromen, stizophyllin, strebloside, akagerine, dihydrousambarensine, hydroxyusambarine, strychnopentamine, strychnophylline, usambarine, usambarensine, daphnoretin, lariciresinol, methoxylariciresinol, syringaresinol, umbelliferon, afromoson, acetylvismione B, desacetylvismione A, vismione A and B, sulfur-containing amino acids including cysteine, salts of the above-illustrated agents, and a combination thereof; the anti-thrombotic agent is selected from among sulfone amide, metronidazol, argatroban, aspirin, abciximab, syntheticantithrombin, bivalirudin, coumadin, enoxaparin, antithrombotics including desulphated and N-reacetylated heparin, tissue plasminogen activator, GpIIb/IIIa platelet membrane receptor, antibodies to factor Xa inhibitor, heparin, hirudin, r-hirudin, PPACK, protamin, sodium 2-methylthiazolidine-2,4-dicarboxylate, prourokinase, streptokinase, warfarin, urokinase, dipyramidole, trapidil, nitroprusside, PDGF antagonists including triazolopyrimidine andseramin, captopril, cilazapril, lisinopril, enalapril, losartan, thio-protease inhibitors, prostacyclin, vapiprost, α-, β- and γ-interferon, histamine antagonist, serotonin blockers, apoptosis inhibitors, p65, NF-kB or Bcl-xL antisense oligonucleotides, halofuginone, nifedipine, tocopherol, vitamin B1, B2, B6 and B12, folic acid, tranilast, molsidomine, tea polyphenol, epicatechin gallate, epigallocatechingallate, Boswellinic acid and derivatives thereof, and a combination thereof.

9. The method according to claim 4, wherein the phenol derivative useful in the present invention is selected from the group consisting of tyramine, hydroxyphenylacetic acid, hydroxypropionic acid, derivatives thereof, and a combination thereof.

10. The method according to claim 4, wherein the catechol derivative is selected from the group consisting of L-dihydroxy phenylalanine (L-DOPA), dopamine, norepinephrine, epinephrine, epigallocatechin gallate, and derivatives thereof, and a combination thereof.

11. The method according to claim 4, wherein the linker is a hydrophilic polymer selected from the group consisting of polyesters, polyanhydrides, polyorthoesters, polyurethanes, polyamides, polypeptides, polynuclear aliphatic hydrocarbons, polynuclear aromatic hydrocarbons, alkyl chains and a combination thereof.

12. The method according to claim 4, wherein the linker is a hydrophilic polymer selected from the group consisting of polyethylene glycol-polylactic acid [PEG-PLA], polyethylene glycol-polycaprolactone [PEG-PCL], polyethylene glycol-poly(DL-lactic-co-glycolic acid)[PEG-PLGA], poly((propylene)fumarate), poly((ethylene)fumarate) and a combination thereof.

13. The method according to claim 4, wherein the linker is a hydrophilic polymer selected from the group consisting of polyethylene glycol[PEG], polyethylene oxide[PEO], polyethylene imine[PEI], polypropylene oxide [PPO], polyvinyl alcohol [PVA], poly(N-isopropylacrylamide)[polyNIPAM], polyfumarate, polyorgano phosphagene, polyacrylic acid [polyAAc], polyacryl sulfonate, polyhydroxyethylmethacrylate [PolyHEMA] and a copolymer thereof.

14. The method according to claim 13, wherein the copolymer is selected from the group consisting of PEO-PPO-PEO (Pluronic® series), 4-armPEO-PPO-PEO (Tetronic® series), PEG-PEI, PEG-PVA, PEG-PEI-PVA, PEI-PVA, poly(NIPAAM-co-AAc), poly(NIPAAM-co-HEMA), and a combination thereof.

15. The method according to claim 1, wherein the polyphenol oxidase is selected from the group consisting of tyrosinase, catechol oxidase and a combination thereof.

16. The method according to claim 1, wherein the substrate is a metal or a polymer.

17. The method according to claim 1, wherein the substrate surface is treated with a solution containing the bioactive molecule and polyphenol oxidase using a method selected from the group consisting of spraying, injecting, painting, immersing, role coating and flow coating.

18. The method according to claim 17, wherein the solution contains the bioactive material at a concentration of from 0.001 to 50 wt % and PPO at a concentration of from 0.001 to 1 KU/ml.

19. The method according to claim 17, wherein the substrate surface is treated for 5 min to 1 hr.

20. A method for immobilizing a bioactive molecule using polyphenol oxidase, comprising:
preparing a polymer having tyramine introduced to both ends thereof (step 1);
treating a substrate surface with the polymer together with polyphenol oxidase to convert the tyramine into a dopaquinone which forms a coordinate bond with the substrate surface, thus anchoring the polymer to the surface (step 2); and
introducing a bioactive molecule into the polymer backbone through a Michael addition reaction or imine formation reaction with the dopaquinone molecule, the bioactive molecule being selected from the group consisting of an anti-proliferative agent, anti-inflammatory agent, an anti-thrombotic agent and a combination thereof (step 3).

21. The method according to claim 20, wherein bioactive molecule is selected from the group consisting of fibroblast growth factor (FGF), vascular endothelial growth factor (VEGF), transforming growth factor (TGF), bone morphogenetic protein (BMP), human growth hormone (hGH), pig growth hormone (pGH), granulocyte colony-stimulating factor (G-CSF), erythropoietin (EPO), macrophage colony-stimulating factor (M-CSF), tumor necrosis factor (TNF), epidermal growth factor (EGF), platelet-derived growth factor (PDGF), interferon-α,β,γ, interleukin-2 (IL-2), calcitonin, nerve growth factor (NGF), growth hormone releasing hormone, angiotensin, luteinizing hormone releasing hormone (LHRH), luteinizing hormone releasing hormone agonist (LHRH agonist), insulin, thyrotropin-releasing hormone (TRH), angiostatin, endostatin, somatostatin, glucagon, endorphine, bacitracin, mergain, colistin, monoclonal antibodies, vaccines, and a combination thereof.

22. The method according to claim 20, wherein the anti-proliferative agent is selected from among sirolimus (rapamycin), everolimus, pimecrolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenphosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, smc proliferation inhibitor-2w, epothilone A and B, mitoxantrone, azathioprine, mycophenolatmofetil, c-myc-antisense, b-myc-antisense, betulinic acid, camptothecin, PI-88 (sulfated oligosaccharide), melanocyte stimulating hormone(α-MSH), activated protein C, IL-1β inhibitors, thymosine α-1, fumaric acid and its esters, calcipotriol, tacalcitol, lapachol, β-lapachone, podophyllotoxin, betulin, podophyllic acid 2-ethylhydrazide, molgramostim (rhuGM-CSF), peginterferon α-2b, lenograstimn (r-HuG-CSF), filgrastim, macrogol, dacarbazine, basiliximab, daclizumab, selectin (cytokine antagonist), CETP inhibitors, cadherines, cytokinin inhibitors, COX-2 inhibitors, NFkB, angiopeptin, ciprofloxacin, fluoroblastin, monoclonal antibodies inhibitive of mycocyte proliferation, bFGF antagonists, probucol, prostaglandin, 1,11-dimethoxycanthin-6-one, 1-hydroxy-11-methoxycanthin-6-one, scopoletin, colchicine, NO donors including pentaerythritol tetranitrate or syndnoeimine, S-nitroso derivatives, tamoxifen, staurosporine, β-estradiol, α-estradiol, estriol, estrone, ethinylestradiol, fosfestrol, medroxyprogesterone, estradiol cypionate, estradiol benzoate, tranilast, cancer therapeutic kamebakaurin and other terpenoids, verapamil, tyrosine kinase inhibitors (tyrphostines), cyclosporine A, paclitaxel and its derivatives such as 6-α-hydroxy-paclitaxel, baccatin, taxotere, macrocyclic oligomers (MCS) of natural or synthetic carbon suboxide and their derivatives, mofebutazone, acemetacin, diclofenac, lonazolac, dapsone, O-carbamoylphenoxyacetic acid, lidocaine, ketoprofen, mefenamicacid, piroxicam, meloxicam, chloroquine phosphate, penicillamine, tumstatin, avastin, D-24851, SC-58125, hydroxychloroquine, auranofin, sodium aurothiomalate, oxaceprol, celecoxib, β-sitosterin, ademetionine, myrtecaine, polidocanol, nonivamide, levomenthol, benzocaine, aescin, ellipticine, D-24851 Calbiochem, colcemid, cytochalasin A-E, indanocine, nocodazole, S 100 protein, bacitracin, vitronectin receptor antagonist, azelastine, guanidyl cyclase stimulator, tissue inhibitor of metal proteinase-1 and -2, free nucleic acids, nucleic acids, DNA and RNA fragments integrated into viral vectors, plasminogen activator inhibitor-1, plasminogen activator inhibitor-2, antisense oligonucleotides, VEGF inhibitors and IGF-1; the anti-inflammatory agent is selected from among natural or synthetic steroids including cefadroxil, cefazolin, cefaclor, cefotaxim, tobramycin, gentamycin, dicloxacillin, oxacillin, leflunomide, anakinra, etanercept, sulfasalazine, etoposide, dicloxacillin, tetracycline, triamcinolone, mutamycin, procainamid, D24851, SC-58125, retinoic acid, quinidine, disopyramide, flecamide, propafenone, sotalol, amidorone, bryophyllin A, inotodiol, maquiroside A, ghalakinoside, mansonine, strebloside, hydrocortisone, betamethasone and dexamethasone, non-steroidal substances (NSAIDS) including fenoprofen, ibuprofen, indomethacin, naproxen and phenylbutazone, antiprozoal agents such as acyclovir, ganciclovir, zidovudine, clotrimazole, flucytosine, griseofulvin, ketoconazole, miconazole, nystatin, terbinafine, chloroquine, mefloquine and quinine, hippocaesculin, barringtogenol-C21-angelate, 14-dehydroagrostistachin, agroskerin, agrostistachin, 17-hydroxyagrostistachin, ovatodiolid, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-β-hydroxypregnadiene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-AII, bisparthenolidine, periplocoside A, ghalakinoside, ursolic acid, deoxypsorospermin, sychorubin, ricin A, sanguinarine, manwu wheat acid, methylsorbifolin, sphatheliachromen, stizophyllin, strebloside, akagerine, dihydrousambarensine, hydroxyusambarine, strychnopentamine, strychnophylline, usambarine, usambarensine, daphnoretin, lariciresinol, methoxylariciresinol, syringaresinol, umbelliferon, afromoson, acetylvismione B, desacetylvismione A, vismione A and B, sulfur-containing amino acids including cysteine, salts of the above-illustrated agents, and a combination thereof; and the anti-thrombotic agent is selected from among sulfone amide, metronidazol, argatroban, aspirin, abciximab, syntheticantithrombin, bivalirudin, coumadin, enoxaparin, antithrombotics including desulphated and N-reacetylated heparin, tissue plasminogen activator, GpIIb/IIIa platelet membrane receptor, antibodies to factor Xa inhibitor, heparin, hirudin, r-hirudin, PPACK, protamin, sodium 2-methylthiazolidine-2,4-dicarboxylate, prourokinase, streptokinase, warfarin, urokinase, dipyramidole, trapidil, nitroprusside, PDGF antagonists including triazolopyrimidine andseramin, captopril, cilazapril, lisinopril, enalapril, losartan, thio-protease inhibitors, prostacyclin, vapiprost, α-, β- and γ-interferon, histamine antagonist, serotonin blockers, apoptosis inhibitors, p65, NF-kB or Bcl-xL antisense oligonucleotides, halofuginone, nifedipine, tocopherol, vitamin B1, B2, B6 and B12, folic acid, tranilast, molsidomine, tea polyphenol, epicatechin gallate, epigallocatechingallate, Boswellinic acid and derivatives thereof, and a combination thereof.
